(19) United States
(12) Patent Application Publication
(10) Pub. No.: US 2021/0311011 A1
(43) Pub. Date: Oct. 7, 2021

(54) TECHNIQUES FOR SCORING FOOD SPECIMENS, AND RELATED METHODS AND APPARATUS

(71) Applicant: TeakOrigin Inc., Boston, MA (US)

(72) Inventors: Brent Darrell Overcash, Boston, MA (US); John Gregory Shewmaker, Woodstock, VT (US); Ellen V. Miseo, Needham, MA (US); Yagiz Sutcu, Arlington, MA (US)

(21) Appl. No.: 17/290,445

(22) PCT Filed: Oct. 30, 2019
(86) PCT No.: PCT/US2019/058963

§ 371 Date: Apr. 30, 2021

Related U.S. Application Data
(60) Provisional application No. 62/752,625, filed on Oct. 30, 2018.

(51) Int. Cl. G01N 33/02; G01N 21/25; G06Q 30/00; G06Q 30/02; G06N 20/00

(52) U.S. Cl. CPC G01N 33/025; G01N 21/25; G06Q 50/28; G06Q 30/0283; G06N 20/00; G06Q 30/0185

(57) ABSTRACT

A method for scoring an aspect of a sample of a food may include obtaining spectroscopic data indicating spectroscopic characteristics of the sample; identifying the food; identifying analytes associated with the aspect of the sample based on the identity of the food; and, for each of the identified analytes, obtaining a measurement model configured to estimate an amount of the analyte present in specimens of the food based on spectroscopic characteristics of the specimens, and using the measurement model to determine an amount of the analyte in the sample based on the spectroscopic data. The method may also include determining a score for the aspect of the sample based on (1) the determined amounts of the identified analytes and (2) reference amounts of the identified analytes and/or reference values of one or more analyte expressions that include combinations of at least two of the identified analytes.

### CROSS-REFERENCE TO RELATED APPLICATIONS

The subject matter of this disclosure is related to subject matter disclosed in U.S. Provisional Patent Application No. 62/752,625 titled “Techniques for Determining Properties of Food Specimens, and Related Methods and Apparatus” and filed on Oct. 30, 2018, which is hereby incorporated by reference herein to the maximum extent permitted by applicable law.

### FIELD OF INVENTION

The present disclosure relates generally to scoring food specimens (or “samples”) based on non-destructive scans (e.g., spectrometric scans) of the specimens.

### BACKGROUND

Modern systems for producing and distributing food generally lack transparency, misrepresent the nutritional quality of the food provided to consumers, and allow significant nutritional value in food specimens to be lost in the production and supply chain. Media coverage often focuses on cases in which food is contaminated with harmful chemicals, pathogens, pesticides, or adulterants. However, even when food is not contaminated, it can be very difficult or impractical for food suppliers and retailers to accurately assess the quality (e.g., nutritional quality) of the food they buy and sell, and the food processing and storage techniques that enable suppliers and retailers to safely and inexpensively distribute food that appears to be fresh can substantially lower the food's quality. Thus, food labels often over-represent the actual nutritional quality of the labeled foods, sometimes to a shocking extent. For all these reasons, it can be difficult or impractical for suppliers (e.g., producers, distributors, and retailers) and consumers of food to (1) accurately assess the quality of the food they produce, buy, sell, store, and/or consume, and (2) trust representations about food quality made by upstream suppliers in the supply chain.

Conventional techniques for assessing food quality, whether destructive or non-destructive, do not provide an adequate solution to the above-described problems. Two of the most common, non-destructive techniques by which suppliers and consumers of food attempt to assess food quality are visual inspection (e.g., visually examining the exterior surface of food specimens) and manual inspection (e.g., physically testing the firmness of food specimens). These techniques can, in some cases, yield a very rough indication of a food specimen's ripeness, but generally do not provide an accurate indication of a food specimen's freshness or quality, in part because these assessment techniques are highly subjective and in part because there is often little or no functional relationship between the visual appearance or firmness of a food specimen and its freshness or quality.

Even destructive techniques for assessing food quality tend to have significant limitations. For example, chemical analysis of food specimens can be performed to identify and quantify the analytes present in the specimens. However, from a practical perspective, such chemical analyses can be performed on only a very small subset of food specimens in the global food supply chain, because chemical analysis processes (1) are generally expensive and time-consuming, and (2) generally render the analyzed specimens unsuitable for consumption.

### SUMMARY

Even when the identities and qualities of the analytes present in a food specimen are known, there are no known techniques for synthesizing this raw physical and/or chemical information into an accurate characterization of the specimen's quality (e.g., nutritional quality) that can be understood and used by participants in the food supply chain to make intelligent decisions about food production, distribution, consumption, and pricing (e.g. “fair pricing”) practices. Thus, there is an urgent need for food quality assessment techniques that can be used by suppliers and/or consumers to quickly and accurately assess the quality of specimens of a wide variety of foods (e.g., at the level of individual specimens, at the level of individual participants in the food supply chain, etc.), and to analyze the impact of food production and supply practices on food quality. The present disclosure describes some embodiments of such food quality assessment techniques. System that perform such techniques may be referred to herein as “food quality assessment systems.”

The present disclosure describes some non-limiting examples of techniques for generating profiles (e.g., “critical composition profiles”) for individual foods. In some embodiments, a food's profile identifies (i) analytes that can be measured to determine the quality of samples of the food and (ii) amounts of those analytes that tend to be present in high-quality samples of the food. For example, the profile for a particular food may identify a relatively small number of analytes that are strong indicators of the quality of samples of the food. Such a profile may also identify the levels (e.g., average levels) of those analytes that are generally observed in a high-quality sample of the food. In some cases, a food's profile may identify contaminants or adulterants associated with the food.

The present disclosure also describes some non-limiting examples of techniques for generating digital datasets for individual foods. In some embodiments, a food's digital dataset includes a profile (e.g., a “critical composition profile”) of the food; sample data (e.g., chemical and/or physical measurements of samples of the food; electromagnetic spectra generated from spectrometric scans of the samples; property data indicating one or more other properties of the samples; etc.); and measurement models (e.g., mathematical, statistical, or machine learning models that can be used to accurately estimate the amounts of the analytes present in samples of the food based on spectral data generated from spectrometric scans of the samples).

The present disclosure also describes some embodiments of techniques for generating measurement models for individual foods based on correlations between (1) spectrometric data indicating spectral characteristics of a set of samples of the food and (2) chemical and/or physical measurements of the amounts of certain analytes present in the samples. Measurement models may be, for example, mathematical, statistical, or machine learning models. In some embodiments, measurement models are trained using machine learning algorithms, with the chemical/physical sample data and the spectral sample data for a suitable set of samples serving as the training and validation data. In some embodiments, measurement models may be used to accurately estimate the amounts of certain analytes present in samples of the food based on spectral data generated from spectrometric scans of the samples. In some cases, suitable spectral data of a sample can be obtained using a brief scan with a field-quality spectrometer, and the measurement model can be used to estimate the analyte levels in the sample based on the spectral data in real time (e.g., within 10 seconds or less).

The present disclosure also describes some embodiments of techniques for generating classification models suitable for accurately identifying a food “class” to which a food specimen belongs based on the specimen's spectral data (e.g., based on attributes of the specimen's identity and/or context derived from the specimen's spectral data). In some embodiments, a classification model may be generated based on correlations between (1) spectroscopic data indicating spectral characteristics of samples of different foods and (2) labels identifying the food classes to which the samples belong. Such labels may, for example, be assigned to the samples by humans. A classification models may include, for example, one or more machine learning classifiers. In some embodiments, a classification model is trained using a machine learning algorithm, with the spectroscopic sample data and the class label data for a suitable set of samples serving as the training and validation data. In some embodiments, a classification model may be used to accurately identify the food classes to which food samples belong based on spectral data generated from spectroscopic scans of the samples. In some cases, suitable spectral data of a sample can be obtained using a brief scan with a field spectrometer, and the classification model can be used to identify the sample's food class based on the spectral data in real time (e.g., within 10 seconds or less).

In some embodiments, a classification model may be used to identify a sample's food class, and then a measurement model specific to the determined food class of the sample may be selected based on the classification, which may facilitate faster and/or more accurate estimation of the amounts of specific analytes in the specimen. If the system is unable to resolve certain attributes of the specimen's identity or context with high confidence, the system may select a measurement model that is more general to a broader food class that includes the specimen but is less specific to the specimen.

The present disclosure also describes some embodiments of techniques for generating predictive models for attributes of individual foods based on correlations between (1) spectroscopic data indicating spectral characteristics of a set of samples of the food and (2) property data indicating values of one or more properties of the samples. In some embodiments, predictive models may be used to accurately estimate certain properties of food samples (e.g., the class of food to which the sample belongs; the region where the sample was produced; the amounts of specific analytes contained in the sample; etc.) based on spectral data generated from spectrometric scans of the samples and/or based on portions of property data. In some cases, suitable spectral data of a sample can be obtained using a brief scan with a field spectrometer, and the predictive models can be used to infer properties of the sample based on the spectral data (and/or portions of property data) in real time (e.g., within 20 seconds or less).

Some examples of properties of food samples may include analytical properties, identity properties, and contextual properties. Analytical properties of the samples may include the measured amounts of one or more analytes in the samples. Identity properties of a sample may include a “class” of the sample, for example, a category, sub-category (“type”), sub-sub-category (“species”), and/or sub-sub-sub-category (“subspecies”) of foods to which the sample belongs. Contextual properties of a sample may include production data (e.g., data identifying a producer of the sample and/or the production practices used to produce the sample), supply data (e.g., data identifying a supplier of the sample and/or supply practices used to load, unload, distribute, store, process, and/or package the sample), retail data (e.g., data identifying a retailer of the sample or retails practices used to unload, store, process, package, and/or present the sample), and/or scoring data (e.g., data indicating scores of aspects of the sample at different stages in the production and distribution chain).

The present disclosure also describes some embodiments of techniques for scoring an aspect of a sample of a food. The scored aspect of the sample may be, for example, the quality of the sample. In some embodiments, the food quality assessment system may (1) use the above-described measurement models to determine the amounts of specific analytes in a sample of a particular food, and (2) assign a score indicating an extent to which an aspect of the sample ‘measures up’ to an objective standard for the quality of samples of that food. In this way, the system can distill a large amount of analytical data into a simple score that entities in the food supply chain (e.g., suppliers, retailers, consumers, etc.) can intuitively understand and easily use to compare different samples and to determine what actions to take with respect to different samples.

In general, one innovative aspect of the subject matter described in this specification can be embodied in a method for scoring an aspect of a sample of a food, including obtaining spectroscopic data indicating spectroscopic characteristics of the sample; obtaining an identity of the food; identifying one or more analytes associated with the aspect of the sample based on the identity of the food and profile data corresponding to the identified food; for each of the identified analytes, obtaining a respective measurement model configured to estimate an amount of the analyte present in specimens of the food based on spectroscopic characteristics of the specimens, and using the respective measurement model to determine an amount of the analyte in the sample based on the spectroscopic data; determining a score for the aspect of the sample based on (1) the determined amounts of the identified analytes in the sample and (2a) respective reference amounts of the identified analytes and/or (2b) respective reference values of one or more analyte expressions, wherein each analyte expression includes a respective combination of at least two of the identified analytes, and wherein the profile data indicate the reference amounts and/or reference values; and presenting the determined score for the aspect of the sample to a user via a user interface of a computer.

Other embodiments of this aspect include corresponding computer systems, apparatus, and computer programs recorded on one or more computer storage devices, each configured to perform the actions of the method. A system of one or more computers can be configured to perform particular actions by virtue of having software, firmware, hardware, or a combination of them installed on the system (e.g., instructions stored in one or more storage devices) that in operation causes or cause the system to perform the actions. One or more computer programs can be configured to perform particular actions by virtue of including instructions that, when executed by data processing apparatus, cause the apparatus to perform the actions.

The foregoing and other embodiments can each optionally include one or more of the following features, alone or in combination. In some embodiments, the aspect of the sample is a quality of the sample. In some embodiments, the score representing the quality of the sample includes a combination of (a) one or more individual analyte scores corresponding to the one or more individual analytes and/or (b) one or more analyte expression scores corresponding to the one or more analyte expressions.

In some embodiments, determining the score representing the quality of the sample includes, for each of the individual analytes: determining a value representing a relationship between the reference amount of the individual analyte and the determined amount of the individual analyte; and determining the individual analyte score corresponding to the individual analyte based on the value representing the relationship between the reference and determined amount of the individual analyte.

In some embodiments, the relationship between the reference and determined amounts of the individual analyte is a ratio of the determined amount to the reference amount, a difference between the determined amount and the reference amount, or a percentage difference between the determined amount and the reference amount. In some embodiments, the individual analyte score corresponding to a particular individual analyte is determined based on a specified function of the value representing the relationship between the reference and determined amounts of the individual analyte. In some embodiments, the specified function includes a linear function, a non-linear function, a parabolic function, an exponential function, and/or a step function.

In some embodiments, determining the score representing the quality of the sample includes, for each of the analyte expressions: combining the determined amounts of the analytes included in the analyte expression according to the combination associated with the analyte expression, thereby obtaining a determined value of the analyte expression; determining a value representing a relationship between the reference value of the analyte expression and the determined value of the analyte expression; and determining the analyte expression score corresponding to the analyte expression based on the value representing the relationship between the reference and determined values of the analyte expression.

In some embodiments, the relationship between the reference and determined values of the analyte expression is a ratio of the determined amount to the reference amount, a difference between the determined amount and the reference amount, or a percentage difference between the determined amount and the reference amount. In some embodiments, the combination of at least two analytes included in a particular analyte expression includes a weighted sum of the at least two analytes, a product of the at least two analytes, a ratio of the at least two analytes, or a specified function of the at least two analytes. In some embodiments, the analyte expression score corresponding to a particular analyte expression is determined based on a specified function of the value representing the relationship between the reference and determined values of the analyte expression. In some embodiments, the specified function includes a linear function, a non-linear function, a parabolic function, an exponential function, and/or a step function.

In some embodiments, each of the individual analyte scores is a numeric value within a range specified for the corresponding individual analyte. In some embodiments, each of the analyte expression scores is a numeric value within a range specified for the corresponding analyte expression. In some embodiments, the combination of the individual analyte scores and/or analyte expression scores is a specified function of the individual analyte scores and/or analyte expression scores. In some embodiments, the specified function of the individual analyte scores and/or analyte expression scores is a weighted linear sum including one or more terms, wherein each of the terms includes a product of (1) a respective term weight and (2) a respective individual analyte score or a respective analyte expression score. In some embodiments, the terms weights are user-adjustable. In some embodiments, the quality score is a numeric value within a specified range. In some embodiments, the quality score is a classification selected from a set of classifications.

In some embodiments, the actions of the method further include making a determination to accept or reject delivery of a shipment of samples of the food including the sample based, at least in part, on the quality score for the sample. In some embodiments, the actions of the method further include accepting or rejecting delivery of the shipment of samples in accordance with the determination.

In some embodiments, the actions of the method further include assigning the sample to a grouping based, at least in part, on the quality score, wherein the grouping is one of a plurality of groupings. In some embodiments, the actions of the method further include placing the sample in a container of samples corresponding to the assigned grouping, wherein the container is one of a plurality of containers corresponding to the plurality of groupings, and wherein the placing is performed by a food-handling machine. In some embodiments, the food-handling machine is a robot, and wherein obtaining the spectroscopic data includes performing a spectroscopic scan of the sample at the plurality of wavelengths using a field spectrometer included in a food-handling component of the robot.

In some embodiments, the actions of the method further include determining a sale price or a purchase price for the sample or a set of samples including the sample based, at least in part, on the quality score for the sample.

In some embodiments, obtaining the identity of the food includes receiving user input indicating the identity of the food. In some embodiments, obtaining the identity of the food includes receiving data obtained by scanning a label associated with the sample of the food, and the identity of the food is determined based on the received data. In some embodiments, obtaining the identity of the food includes classifying the sample based on at least a portion of the spectroscopic data indicating the spectroscopic characteristics of the sample, wherein classifying the sample includes: providing at least the portion of the spectroscopic data as input to a classifier; and executing the classifier on the provided input, wherein the classifier provides output indicating a classification of the sample, and wherein the classification indicates the identity of the food.

In some embodiments, the identified analytes include four or more analytes selected from a group including at least one sugar, at least one acid, at least one vitamin, at least one mineral, at least one fat, at least one starch, at least one fiber, at least one carotenoid, at least one flavonoid, at least one protein, moisture content, alcohol content, and/or gluten.

In some embodiments, the food is an apple and the identified analytes include sucrose, glucose, fructose, malic acid, ascorbic acid, moisture content, anti-oxidants, and/or total anthocyanins. In some embodiments, the food is a blueberry and the identified analytes include glucose, fructose, moisture content, and/or total anthocyanins. In some embodiments, the food is a banana and the identified analytes include sucrose, glucose, fructose, malic acid, citric acid, ascorbic acid, and/or moisture content. In some embodiments, the food is a green grape and the identified analytes include malic acid, tartaric acid, moisture content, glucose, and/or fructose. In some embodiments, the food is a red grape and the identified analytes include malic acid, tartaric acid, moisture content, glucose, fructose, and/or total anthocyanins. In some embodiments, the food is a tomato and the identified analytes include lycopene, malic acid, citric acid, ascorbic acid, moisture content, glucose, fructose, and/or total carotenoids. In some embodiments, the food is a strawberry and the identified analytes include glucose, fructose, ascorbic acid, total anthocyanins, citric acid, anti-oxidants, and/or moisture content. In some embodiments, the food is spinach and the identified analytes include moisture content, ascorbic acid, anti-oxidants, oxalic acid, total carotenoids, and Lutein carotenoids. In some embodiments, the food is avocado and the identified analytes include moisture content, lipids, linoleic fatty acid, oleic fatty acid, palmitic fatty acid, and/or palmitoleic fatty acid.

In some embodiments, the food is a fruit and the identified analytes include moisture content; at least one sugar selected from the group consisting of glucose and fructose; and at least one acid selected from the group consisting of ascorbic acid and malic acid.

In some embodiments, obtaining the spectroscopic data includes performing a spectroscopic scan of the sample at the plurality of wavelengths. In some embodiments, the spectroscopic scan is performed by a field spectrometer. In some embodiments, the field spectrometer is hand-held, coupled to an automated food-handling device, or coupled to an automated food-distribution device.

In some embodiments, the measurement model is a linear multivariate regression model, a non-linear multivariate regression model, or a blend of two or more of the foregoing. In some embodiments, the aspect of the sample is a quality-price index of the sample, wherein the score for the aspect of the sample is a quality-price index score, and wherein the quality-price index score is further based on a price of the sample. In some embodiments, the quality-price index score is based on a ratio between a price of the sample and a quality score of the sample.

Particular embodiments of the subject matter described in this specification can be implemented so as to realize one or more of the following advantages:

(1) By assigning a score that represent the extent to which an aspect of a food sample (e.g., the sample's overall quality, ripeness, spoilage, freshness, etc.) achieves a target status (e.g., a target status specified by a user or associated with an objective standard), the food quality assessment system can distill a large amount of analytical data into a simple score that entities in the food supply chain (e.g., suppliers, retailers, consumers, etc.) can intuitively understand and easily use to compare different samples and to determine whether to purchase or consume a sample.

(2) By permitting user-controllable customization of the scoring criteria, the food quality assessment system can enable different entities in the food supply chain to specify the extent to which different properties of a food are important to the entity, while still distilling the analysis of each sample into a simple, easy-to-understand score.

(3) Entities in the food supply chain can use scoring data for food samples to make real-time decisions related to their participation in the supply chain based on accurate data regarding the quality of samples. For example, a retailer or supplier can accept or reject delivery of a food shipment based on the scores for samples in the shipment; a consumer can decide which samples to purchase and how much to spend on the samples based on their scores; a supplier or retailer can adjust the operating parameters of a food storage facility or container based on how the facility or container has affected the scores of previously stored samples; a supplier or retailer can sort samples into bins according to their samples' quality scores; etc. By providing such scoring data, the food quality assessment system can improve users' capacity to identify, buy, sell, and/or consume higher-quality foods rather than lower-quality foods.

(4) By checking spectrometric scans of samples for evidence of contaminants or adulterants, the food quality assessment system can improve users' ability to detect and avoid contaminated or adulterated samples, thereby improving health outcomes for users.

(5) The fidelity or predictive power of spectroscopic data obtained using low-resolution spectrometers (e.g., field spectrometers) can be enhanced.

(6) The food quality assessment system can be used to obtain longitudinal analytical data and scoring data for food samples as the samples move through the food production and distribution chain. The system can analyze this longitudinal data to determine how various stages in the food production and distribution chain tend to affect the quality of the food samples that pass through those stages. Thus, the system may provide users with an improved capacity to identify (1) producers, suppliers, and retailers that tend to provide high-quality food samples, and (2) production practices, supply practices, and retail practices that tend to produce high-quality (or low-quality) food samples and/or preserve (or degrade) food quality. In this way, embodiments of the system can enable users to purchase food samples having a desired level of quality, which can provide strong incentives for producers, suppliers, and retailers to adjust their food production, supply, storage, processing, and packaging processes to provide food samples have the desired level of quality.

The details of one or more embodiments of the subject matter described in this specification are set forth in the accompanying drawings and the description below. Other features, aspects, and advantages of the subject matter will become apparent from the description, the drawings, and the claims.

The foregoing Background and Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

Certain advantages of some embodiments may be understood by referring to the following description taken in conjunction with the accompanying drawings. The drawings are not necessarily to scale, emphasis instead generally being placed upon illustrating principles of some embodiments of the invention.

FIG. 1 is a flowchart of a method for generating a digital dataset for a food, according to some embodiments.

FIG. 2 is a flowchart of an exemplary method for the analysis of a batch of apples as described in Example 1.

FIG. 3 is a flowchart of a family of machine learning methods for developing measurement models that can be used to accurately estimate the amounts of the analytes present in samples of foods based on spectral data generated from spectrometric scans of the samples, according to some embodiments.

FIG. 4 is shows diagrams of exemplary field-grade spectrometers.

FIG. 5 is a flowchart of another method for developing measurement models that can be used to accurately estimate the amounts of the analytes present in samples of foods based on spectral data generated from spectrometric scans of the samples, according to some embodiments.

FIG. 6 is a flowchart of another method for developing measurement models that can be used to accurately estimate the amounts of the analytes present in samples of foods based on spectral data generated from spectrometric scans of the samples, according to some embodiments.

FIG. 7 shows regression charts which illustrate the correlation between estimates of analyte values provided by some embodiments of measurement models for a set of food samples and laboratory measurements of the analyte values in the same samples, as described in Example 1.

FIG. 8 is a flowchart of a method for scoring an aspect of a sample of a food, according to some embodiments.

FIG. 9 is a block diagram of an implementation of a computer system.

### DETAILED DESCRIPTION

The present disclosure describes, among other things, techniques for scoring aspects of samples of food, including but not limited to techniques for scoring the quality of a food sample. To facilitate the scoring of the quality of a sample of a food, a digital dataset for the food may be generated. In some embodiments, a food's digital dataset includes a food profile (e.g., a “critical composition profile”) that identifies (i) analytes that can be measured to determine the quality of samples of the food and (ii) amounts of those analytes that tend to be present in high-quality samples of the food; sample data (e.g., chemical and/or physical measurements of samples of the food; electromagnetic spectra generated from spectrometric scans of the samples; property data indicating one or more other properties of the samples; etc.); and measurement models (e.g., mathematical, statistical, or machine learning models that can be used to accurately estimate the amounts of the analytes present in samples of the food based on spectral data generated from spectrometric scans of the samples). Some embodiments of techniques for generating digital datasets are described below in the section titled “Generation of Digital Datasets for Foods.”

### Generation of Digital Datasets for Foods

In some embodiments, the present disclosure contemplates, among other things, generation of a digital dataset for a food (e.g., a cultivar). In some embodiments, a digital dataset is a digital representation of a food. In some embodiments, a digital dataset can be used to assess a food sample. In some embodiments, assessment of a food sample includes, among other things, determining its quality.

Some examples of foods are described below in the subsection titled “Foods.”

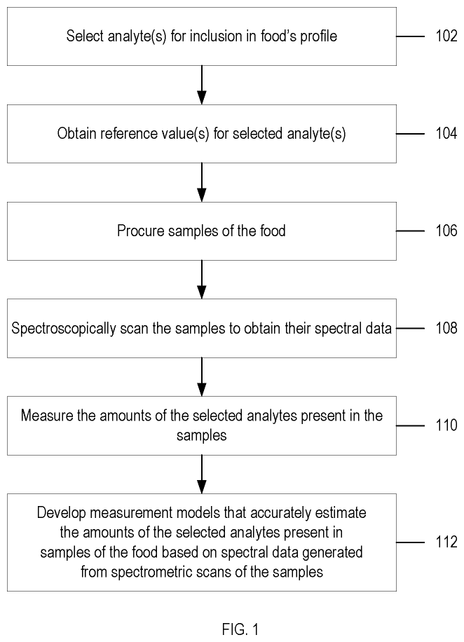

Some embodiments of the techniques described in the present disclosure are suitable for generation of a digital dataset for a food (e.g., a single-ingredient food). Referring to FIG. 1, a method 100 for generating a digital dataset for a food may comprise steps of selecting (102) one or more analytes for inclusion in the food's profile; obtaining (104) reference values for the selected analytes; procuring (106) samples of the food; spectroscopically scanning (108) the samples to obtain their spectral data; measuring (110) the amounts (e.g., quantities or concentrations) of the selected analytes present in the samples (e.g., using lab-based analytical chemistry protocols and methods); and developing (112) measurement models that can accurately estimate the amounts of the analytes present in samples of the food based on spectral data generated from spectrometric scans of the samples. Some embodiments of the steps of the method 100 are described in further detail below.

In step 102, one or more analytes are selected for inclusion in the food's profile (e.g., “critical composition profile” or “CCP”). The present disclosure describes some embodiments of techniques for generating profiles for different foods (e.g., different cultivars). The profile for a particular food may identify a relatively small number (e.g., 3-10, fewer than 15, fewer than 20, etc.) of parameters (e.g., chemical or physical properties, which may be referred to herein as “analytes”) that are strong indicators of the quality of samples of the food. The profile for a particular food may also identify the reference levels (e.g., average levels) of those analytes that are generally observed in a high-quality sample of the food. In some embodiments, reference levels of a food's analytes may be identified in published nutritional standards (e.g., the U.S. Department of Agriculture's nutritional standards) or other published works (e.g., peer reviewed literature). In some cases, a food's profile may identify contaminants or adulterants associated with the food.

In some embodiments, a food's profile identifies three or more parameters of the food that can be measured in samples of the food and analyzed to assess the quality of those samples. The parameters of a food identified in the food's CCP are sometimes referred to herein as the food's “analytes” or “critical analytes.” In some embodiments, a food's critical composition profile may identify one or more specific interrelationship(s) between analytes that are indicative of the quality of the food and/or changes in the quality of the food over time. In some embodiments, the parameters are detectable with spectroscopic techniques or are highly correlated with other properties of the food that can be detected with spectroscopic techniques.

Any suitable criteria may be used to select analytes for a food's profile from a set of candidate analytes, including but not limited to chemical and spectroscopic detection limits associated with the candidate analytes; the importance of the candidate analytes as indicators of changes in the food; public, consumer, and/or industrial interest in the candidate analytes; and/or the relationship(s) between the candidate analyte and other analytes. These selection criteria are described in further detail below.

Chemical and spectrometric detection limits: A person of ordinary skill in the art will understand that certain analytical techniques have their own detection limits. As used herein, the chemical “limit of detection” refers to the smallest amount of the analyte that can be detected using the measurement techniques described below with reference to step 110, and the spectrometric “limit of detection” refers to the smallest amount of the analyte that can be detected and estimated using the spectrometric techniques described below with reference to step 108 and the measurement models described below with reference to step 112. Analytes of a particular food with natural concentration levels that do not fall within these detection limits (e.g., are lower than the detection limits) are generally not suitable for inclusion within a food's profile.

Importance of an analyte as an indicator of changes in a food: For a given food, the concentration of some analytes may change in predictable ways as samples of the food grow and before they are harvested but may remain relatively stable after harvesting. Such analytes may be good indicators of the growing and harvesting practices applied to the samples, and therefore may be generally suitable for inclusion within the food's profile. For example, malic acid is relatively stable in strawberries after they are harvested and is therefore generally a good indicator of growing and harvesting practices used for strawberries.

The concentration of other analytes may increase or decrease (significantly in some cases) after the harvest, while the food is stored and shipped. Such analytes may be good indicators of a food's freshness, and therefore may be generally suitable for inclusion within the food's profile. For example, ascorbic acid (Vitamin C) is generally very delicate in strawberries, in the sense that Vitamin C increases as strawberries ripen on the plant but degrades over time while the berries are shipped and stored. Thus, Vitamin C is a generally a good indicator of the freshness of strawberries.

Public, consumer, and industrial interest in the analyte: Consumers, producers, and sellers of food may express interest in certain analytes for reasons of taste and/or nutritional values, or for other reasons (including but not limited to historical reasons). For example, analytes with antioxidant properties, such as ascorbic acid and anthocyanins, are often important to consumers, giving antioxidant packed fruits higher commercial value. In general, greater interest in an analyte may translate to greater suitability of the analyte for inclusion within a food's profile.

Relationship of the analyte to other analytes: Relationships between (or among) analytes can affect the suitability of one or more of the related analytes for inclusion in a food's profile. For example, in some cases, relationships between a spectroscopically difficult-to-measure analyte (e.g., an analyte that tends to be present in amounts below the spectroscopic limit of detection) and a spectroscopically easy-to-measure analyte (e.g., an analyte that tends to be present in amounts above the spectroscopic limit of detection) may allow for the amount of the difficult-to-measure analyte to be accurately and reliably estimated using spectral data and a measurement model. In such cases, the difficult-to-measure analyte may be included in the food's profile, and the amount of the difficult-to-measure analyte may be estimated, for example, based on the estimated amount of the easy-to-measure analyte and the correlation between the amounts of the easy-to-measure and difficult-to-measure analytes.

As another example, relationships among analytes within an analyte class may increase the suitability of the analyte class for inclusion in a food's profile. Some interesting analytes in produce are generally not present at significantly high levels. However, many classes of compounds have very similar structures, and the spectra for those compounds are generally very similar. Therefore, over a small range of wavelengths, all the compounds in a chemical class generally exhibit absorbance based on the similar chemical structures. For example, in strawberries, anthocyanins and antioxidants encompass a class of various, similar analytes. For such compounds and chemical classes, analysis methods that detect the amount of the class of compounds are generally able to accurately predict the overall amount of the analyte family, whereas analysis techniques that detect the amounts of the individual analytes and aggregate those amounts to generate an estimate of the amount of the class of compounds (e.g., chromatographic methods) are generally less accurate. In general, an analyte class may be suitable for inclusion in a food's profile if the analyte class includes one or more analytes of interest (e.g., analytes that meet other inclusion criteria), the analyte(s) of interest are individually difficult to measure, the amounts of the analyte(s) of interest are generally highly correlated with the amount of the analyte class, and the amount of the analyte class is measurable within the system's limits of detection.

Some non-limiting examples of analytes that may be selected as critical analytes for various foods are described below in the subsection titled “Analytes.”

Still referring to FIG. 1, in step 104 reference values for selected analytes of a food are obtained. A person of ordinary skill in the art will appreciate that reference values of analytes can be extracted from any suitable source including, for example, USDA nutrition standards. In addition or in the alternative, other techniques, including but not limited to averaging analyte values reported by industry experts or in relevant literature (e.g., peer-reviewed academic publications), may be used as to obtain reference values. Such reference values may be used in place of reference values derived from USDA nutrition standards or when USDA nutrition standards are not available for an analyte. Some non-limiting examples of reference values for analytes that may be selected as critical analytes for various foods are described below in the subsection titled “Reference Values for Analytes.”

Still referring to FIG. 1, in step 106 samples of the food are procured. A number of factors may contribute to the selection of samples. In general, a large quantity of samples (e.g., 100-1000 samples or more) is preferred. In some embodiments, the initial quantity of samples is 100. In addition, a person of ordinary skill in the art will appreciate that procuring a large number of samples of the food having a wide variety of properties (e.g., an accurate sample of the universe of specimens of the food) generally leads to a more robust digital dataset, which in turn leads to the development of improved (e.g., more accurate) measurement models for the food's analytes. For example, variation among one or more of the following attributes within the procured samples may lead to more robust digital datasets and more accurate measurement models: the origin of the sample (e.g., the region in which the sample was grown, the farmer who grew the sample, the date when the sample was harvested, etc.), the point in the food supply and distribution chain at which the sample was collected (e.g., at the farm immediately after harvest, at the farm after harvest and storage, after shipment to a warehouse, after storage in a warehouse, after shipment to a retailer, etc.), the quality of the sample, the amount(s) of one or more critical analytes in the sample, and/or any other suitable attribute of the sample.

Still referring to FIG. 1, in step 108 the samples are spectroscopically scanned to obtain their spectral data. Any suitable type of spectroscopic analysis may be performed, including (without limitation), near-infrared spectroscopy (NIR), ultraviolet-visible spectroscopy (UV/VIS), visible spectroscopy (VIS), and/or Raman spectroscopy. A person of ordinary skill in the art will appreciate that detection limits for near-infrared spectroscopy may be, at best, 0.1% by weight of a material.

In step 108, the spectroscopic analysis may be performed using any suitable type of spectroscopic equipment and/or technique, including (without limitation) laboratory-quality spectroscopic equipment and/or techniques. In some embodiments, a laboratory-grade spectroscopic scan is performed using any suitable device, including but not limited to a PerkinElmer Spectrum Two MIR. In some embodiments, electromagnetic spectra are measured in a range selected from the group consisting of 1100-1700 nm, 200-1050 nm and 400-2500 nm. In some embodiments, electromagnetic spectra are measured in a range selected from the group consisting of 2500-22000 nm, 1000-2500 nm and 200-2200 cm⁻¹.

In addition, or in the alternative, the spectroscopic analysis in step 108 may be performed using field-quality spectroscopic equipment and/or techniques. Relative to “laboratory-quality” spectroscopic scanners, field-quality spectroscopic scanners are generally smaller (e.g., handheld), less expensive, easier to repair and/or replace, and more robust. In addition, relative to laboratory-quality scanners, field-quality scanners generally provide lower-resolution scan data.

In some embodiments, a field-quality spectroscopic technique is a combination of field-quality VIS and field-quality NIR, referred to herein as “field-quality VIS+NIR.” In some embodiments, a field-quality device suitable for performing field-quality VIS+NIR is Malvern Panalytical ASD Quality Spec. In some embodiments, a field-quality VIS+NIR device uses a spectroscopic range of 400-1700 nm.

In some embodiments, a field-quality spectroscopic technique is field-quality NIR. In some embodiments, a field-quality device suitable for performing field-quality NIR is a ThermoFisher Scientific microPHAZIR. In some embodiments, a field-quality NIR device uses a spectroscopic range of 1100-1700 nm.

In some embodiments, a field-quality spectroscopic technique is a field-quality-Raman. In some embodiments, a field-quality device suitable for performing field-quality Raman is a B&WTek iRaman Plus. In some embodiments, a field-quality Raman device uses a spectroscopic range of 200 to 4200 cm⁻¹.

In some embodiments, a field-quality spectroscopic technique is VIS. In some embodiments, a field-quality device suitable for performing a field-quality VIS spectroscopic scan is a B&W Tek Exemplar X Vis. In some embodiments, a field-quality VIS device uses a spectroscopic range of 350-1050 nm.

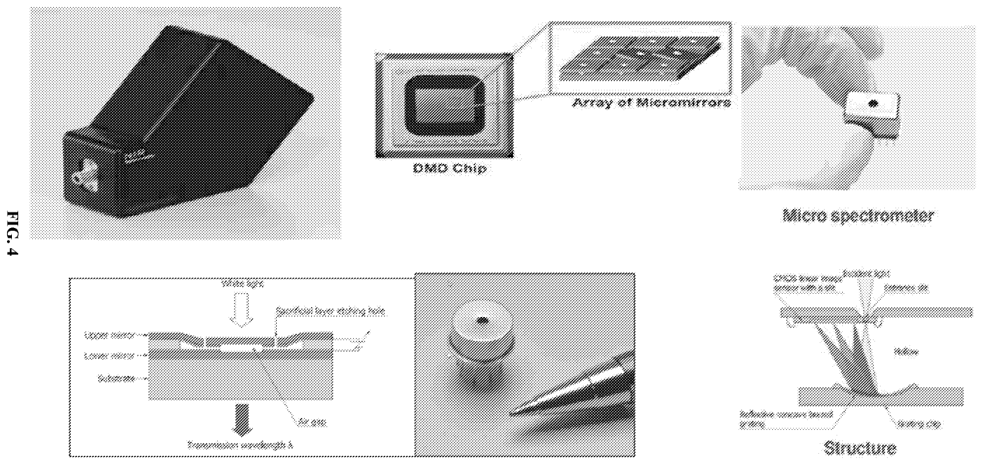

Some examples of field-quality spectroscopic scanners are shown in FIG. 4. A non-limiting example of a data collection protocol that can be used to perform the spectroscopic analysis of the samples in step 108 is described below in the subsection titled “Spectroscopic Data Collection.”

In step 110, the amounts of the selected analytes present in the samples are measured (e.g., using laboratory-based analytical chemistry techniques). A food sample may be analyzed without alteration (e.g., without pre-processing of the sample), or a food sample may be processed prior to analyzation. Analysis of a food may provide, among other things, profile(s) of one or more properties selected from the group consisting of Brix, titratable acidity, starch, sugar, and acid. For example, a food may be blended, and the resultant mash may be analyzed. In some embodiments, the mash is analyzed using thermogravimetric analysis (TGA). In some embodiments, laboratory-grade TGA analysis is performed using any suitable device, including but not limited to a PerkinElmer Pyris 1 TGA. In some embodiments, the pH of a mash of a food can be measured. Alternatively, or in addition, juice of a sample food may be analyzed. In such an example, juice of a food may be analyzed using Ultra Performance Liquid Chromatography (UPLC). UPLC may be useful to determine an acid and/or sugar profile of juice of a food. In some embodiments, UPLC is performed using a PerkinElmer Altus A-30 UPLC. Additionally, or in the alternative, juice of a food may be analyzed using a refractometer to provide a refractive index. In some embodiments, a refractometer is an Atago N-10 Refractometer. In some embodiments, a food sample is analyzed using thermogravimetric analysis (TGA), which may be performed using a PerkinElmer Pyris 1 TGA.

In step 112, measurement models for the food are generated. Some embodiments of these measurement models can be used to accurately estimate the amounts of the analytes present in samples of the food based on spectral data generated from spectroscopic scans of the samples. Together, the analyte measurements and the spectral data for the samples (collected in steps 110 and 108, respectively) can be used as training and validation data to train mathematical, statistical, or machine learning models to estimate the quantity of an analyte present in a sample based on the sample's spectral data, and to validate the accuracy of the trained models.

The measurement models can be trained using any suitable technique. Three such model-training techniques are described below. For convenience, these training techniques and the resulting models are referred to herein as Type-1, Type-2, and Type-3 training techniques and models.

Type-1 training techniques and models: In some embodiments, the measurement models are trained by correlating laboratory-quality spectral data directly with analyte measurements. In other words, a type-1 measurement model may be trained to estimate an analyte measurement for a sample based on the sample's laboratory-quality spectral data.

Type-2 training techniques and models: In some embodiments, the measurement models are trained by correlating field-quality spectral data directly with analyte measurements. In other words, a type-2 measurement model may be trained to estimate an analyte measurement for a sample based on the sample's field-quality spectral data.

Type-3 training techniques and models: In some embodiments, the measurement models are trained by (a) correlating field-quality spectra with laboratory-quality spectral data, and (b) correlating laboratory-quality spectral data with analyte measurements. In other words, a type-3 measurement model may include two constituent models, which are referred to herein for convenience as a stage-1 model and a stage-2 model. The stage-1 model may be trained to estimate a sample's laboratory-quality spectral data based on the sample's field-quality spectral data, and the stage-2 model may be trained to estimate an analyte measurement for a sample based on the output of the stage-1 model (e.g., the sample's estimated laboratory-quality spectral data).

Some non-limiting examples of training techniques that can be used to generate suitable measurement models in step 108 are described below in the subsection titled “Generating Measurement Models.”

Some embodiments of each of the types of training techniques described herein are suitable for developing a set of measurement models suitable for accurately estimating the amounts of the selected analytes present in samples of a food based on spectroscopic scans of the samples. Some embodiments of types of measurement models described herein are suitable for accurately estimating the amounts of the selected analytes present in samples of a food based on spectroscopic scans of the samples.

In some embodiments, the method 100 may be performed iteratively until the selected analytes are determined to be indicative of the quality of samples of the food and the measurement models are determined to provide sufficiently accurate estimates of the amounts of those analytes present in samples of the food based on spectrometric scans (e.g., field-quality spectrometric scans) of the samples. In some embodiments, a measurement model is sufficiently accurate if the model's root mean square error of prediction (RMSEP) is less than or equal to a threshold RMSEP value and the model's correlation coefficient (R) is greater than or equal to a threshold R value. One of ordinary skill in the art will appreciate that, during any iteration of the method 100 for generating a food's digital dataset, the steps of the method may be performed sequentially in the order shown in FIG. 1 or in an order other than the order shown in FIG. 1, with one or more steps repeated and/or omitted.

For example, in an initial iteration of the method 100, a set of analytes for a food may be selected (step 102), reference values for the selected analytes may be obtained (step 104), samples of the food may be procured (step 106), the samples may be spectroscopically scanned (e.g., using laboratory-quality instruments and/or field-quality instruments) (step 108), amounts of analytes present in the food may be measured (step 110), and measurement models may be generated (112). At step 112, a determination may be made that the measurement model(s) for one or more of the selected analytes are not sufficiently accurate. In that case, step 112 may be repeated one or more times until sufficiently accurate measurement models for all analytes have been developed. If the measurement model for an analyte is still not sufficiently accurate after repeating step 112 for at least a threshold time period or for at least a threshold number of iterations, the method 100 may return to step 106 (more samples may be obtained) and proceed again through steps 108-112.

Alternatively, a determination may be made that developing sufficiently accurate measurement models for one or more of the analytes is impractical. In this case, the difficult-to-model analytes may be removed from the critical composition profile, and one or more new analytes may be selected for the critical composition profile in a second iteration of step 102. If the newly-selected analytes were already measured during the initial iteration of step 110, then additional iterations of steps 104-110 may be unnecessary, and the method may proceed directly to or more additional iterations of step 112, in which measurement model(s) for the newly-selected analyte(s) may be developed. On the other hand, if the newly-selected analytes were not already measured during the initial iteration of step 110, then additional iterations of steps 104-110 may be performed, such that spectroscopic scans of new samples are obtained (step 108), the amounts of the analytes (e.g., all the selected analytes or just the newly-selected analytes) present in the new samples are measured (step 110), and measurement models for the analytes (e.g., all the selected analytes or just the newly-selected analytes) are developed using the new data.

An embodiment has been described in which performing the method 100 produces (1) a critical composition profile for a food with analytes A_{Q} that indicate the “quality” of samples of the food, and (2) a digital dataset for the food with measurement models that can accurately estimate the amounts of those analytes A_{Q} present in samples of the food based on the samples' spectral data. As will be described in further detail below, the quality of the samples can then be “scored” based on the relationship between the estimated amounts of those analytes A_{Q} in the samples and the reference amounts of those analytes for the food.

However, foods have properties other than “quality,” and some embodiments of the method 100 may be used to produce (1) a critical composition profile for a food with analytes A_{P} indicative of another property P of samples of the food, and (2) a digital dataset for the food with measurement models that can accurately estimate the amounts of those analytes A_{P} present in samples of the food based on the samples' spectral data. The techniques described below may then be used to “score” that property P of samples of the food based on the relationship between the estimated amounts of those analytes A_{P} in the samples and the reference amounts of those analytes A_{P} for the food.

In some embodiments of the method 100, digital datasets for one or more other properties of a food may be generated. In some cases, the critical composition profile for a property P of a food other than “quality” may include one or more (e.g., all) of the analytes A_{Q} that indicate the quality of the food. In such cases, the reference values associated with those analytes for the purpose of assessing the food's property P may be the same or may differ from the reference values associated with those analytes for the purpose of assessing the food's “quality.” In some cases, the critical composition profile for a property P of a food other than “quality” may include one or more other analytes A_{P} that are indicative of the property P of the food and are not included in the CCP for the food's quality.

Some examples of other properties of food for which digital datasets can be generated using embodiments of the method 100 may include, without limitation, the provenance, age, freshness, adulteration, moisture, acidity, protein, starch, fat, sugars, alcohol content, vitamins, spoilage, ripeness, texture, dilution, dyes, contamination, gluten, fiber, or origin of samples of the food.

Some embodiments of techniques for detecting contaminants in food samples are described below in the subsection titled “Detecting Contaminants.”

### Foods

In some embodiments, foods may be classified into categories, categories of foods may be classified into types of foods, types of foods may be classified into species of foods, and species of foods may be classified into subspecies of foods.

In some embodiments, a category of the food is (1) fruit, (2) vegetable, (3) legume or bean, (4) grain, (5) protein, (6) dairy, or (7) liquid.

In some embodiments, a category of food is fruit and a type of fruit is açai berry, apple, akee, apricot, avocado, banana, bilberry, blackberry, black currant, black sapote, blueberry, boysenberry, crab apple, currant, cherry, cherimoya, chico fruit, cloudberry, coconut, cranberry, cucumber, damson, date, dragon fruit, durian, elderberry, feijoa, fig, fingered citron, goji berry, gooseberry, grape, raisin, grapefruit, guava, honeyberry, huckleberry, jabuticaba, jackfruit, jambul, Japanese plum, jostaberry, jujube, juniper berry, kiwano, kiwifruit, kumquat, lemon, lime, loquat, longan, lychee, mango, mangosteen, Marion blackberry, melon, cantaloupe, honeydew, watermelon, miracle fruit, mulberry, nectarine, nance, olive, orange, blood orange, clementine, mandarin orange, tangerine, papaya, passionfruit, peach, pear, persimmon, plantain, plum, prune, pineapple, pineberry, plumcot, pomegranate, pomelo, purple mangosteen, quince, raspberry, salmonberry, rambutan, redcurrant, salal berry, salak, satsuma, soursop, star apple, star fruit, strawberry, surinam cherry, tamarillo, tamarind, ugli fruit, yuzu, white currant, or white sapote. In some embodiments, the type of fruit is apple, and the species of apple is Red Delicious, Gala, Golden Delicious, Granny Smith, Fuji, McIntosh, Rome, Empire, Honeycrisp, Idared, Jonathan, York, Cripps Pink, Braeburn, Cortland, Northern Spy, Jonagold, Stayman, Pink Lady, or Cameo.

In some embodiments, a category of the food is vegetable, and a type of the vegetable is amrud, artichoke, asparagus, avocado, broccoflower, broccoli, Brussel sprout, cabbage, kohlrabi, cauliflower, celery, corn, cucumber, endive, eggplant, fiddlehead, frisee, fennel, greens, herb or spice, lettuce, arugula, mushroom, nettle, okra, olive, onion, parsley, pepper, radicchio, rhubarb, root vegetable, salsify, skirret, sweetcorn, topinambur, squash, tat soi, tomato, tuber, water chestnut, or watercress. In some embodiments, the type of vegetable is greens, and the species of greens is beet greens, bok choy, chard, collard greens, kale, mustard greens, spinach, or quinoa. In some embodiments, the type of vegetable is herb or spice, and the species of herb or spice is anise, basil, caraway, cilantro, coriander, chamomile, dill, fennel, lavender, lemon grass, marjoram, oregano, parsley, rosemary, sage, or thyme. In some embodiments, the type of vegetable is onion, and the species of onion is chives, garlic, leek, shallot, or scallion. In some embodiments, the type of vegetable is pepper, and the species of pepper is bell pepper, chili pepper, jalapeno pepper, habanero, paprika, tabasco pepper, or cayenne pepper. In some embodiments, the type of vegetable is root vegetable, and the species of root vegetable is beet, carrot, celeriac, daikon, ginger, parsnip, rutabaga, turnip, radish, wasabi, horseradish, or white radish. In some embodiments, the type of vegetable is squash, and the species of squash is acorn squash, bitter melon, butternut squash, banana squash, zucchini, cucumber, delicata, gem squash, hubbard squash, marrow, patty pans, pumpkin, or spaghetti squash. In some embodiments, the type of vegetable is tuber, and the species of tuber is jicama, Jerusalem artichoke, potato, quandong, sunchoke, sweet potato, taro, or yam.

In some embodiments, the category of the food is legume or bean, and the type of legume or bean is alfalfa sprout, adzuki bean, bean sprout, black bean, black-eyed pea, borlotti bean, broad bean, chickpea, green bean, kidney bean, lentil, lima bean, mung bean, navy bean, pinto bean, runner bean, split pea, soy bean, pea, or snap pea.

In some embodiments, the category of the food is grain, and the type of grain is warm-season cereal, cool-season cereal, or pseudocereal grain. In some embodiments, the type of grain is warm-season cereal, and the species of warm-season cereal is finger millet, fonio, foxtail millet, Japanese millet, Job's tears, kodo millet, maize, millet, pearl millet, proso millet, or sorghum. In some embodiments, the type of grain is cool-season cereal, and the species of cool-season cereal is barley, oats, rice, rye, spelt, teff, triticale, wheat, or wild rice. In some embodiments, the type of grain is pseudocereal grain, and the species of pseudocereal grain is amaranth, buckwheat, chia, quinoa, kañiwa, or kiwicha.

In some embodiments, the category of the food is protein, and the type of protein is beef, lamb, eggs, pork, poultry, seafood, or soy. In some embodiments, the type of protein is beef, and the species of beef is chuck, shank, brisket, rib, short plate, flank, loin, sirloin, or round. In some embodiments, the type of protein is poultry, and the species of poultry is chicken, Cornish game hen, duck, emu, goose, grouse, guinea hen, ostrich, partridge, pheasant, quail, squab, or turkey. In some embodiments, the type of protein is seafood, and the species of seafood is fish, shellfish, or roe.

In some embodiments, the species of seafood is fish, and the subspecies of the fish is anchovy, basa, bass, black cod, blowfish, bluefish, Bombay duck, bream, brill, butter fish, catfish, cod, dogfish, dorade, eel, flounder, grouper, haddock, hake, halibut, herring, Ilish, John Dory, kingfish, lamprey, lingcod, mackerel, mahi, monkfish, mullet, orange roughy, parrotfish, Patagonian toothfish, pike, pilchard, pollock, pomfret, pompano, sablefish, salmon, sanddab, sardine, sea bass, shad, shark, skate, smelt, snakehead, snapper, sole, sprat, suiter-fish, sturgeon, surimi, swordfish, tilapia, tilefish, trout, tuna, albacore tuna, yellowfin tuna, bigeye tuna, bluefin tuna, turbot, wahoo, whitefish, or whiting. In some embodiments, the species of seafood is shellfish, and the subspecies of the shellfish is crab, Dungeness crab, mud crab, sand crab, king crab, snow crab, crayfish, lobster, American lobster, rock lobster, spiny lobster, red lobster, shrimp, mollusks, cockle, cuttlefish, loco, mussel, octopus, oyster, periwinkle, scallop, bay scallop, sea scallop, squid, or escargot. In some embodiments, the species of seafood is roe, and a subspecies of the roe is caviar, Beluga caviar, Ossetra caviar, Sevruga caviar, Sterlet caviar, Kaluga hybrid caviar, white sturgeon caviar, Siberian sturgeon caviar, sturgeon roe, paddlefish roe, bowfin roe, whitefish roe, trout roe, salmon roe, steelhead roe, lumpfish roe, whitefish roe, carp roe, ikura, tobiko, masago, uni, black lumpfish roe, or tuna bottarga.

In some embodiments, the category of the food is dairy, and the type of dairy is milk, butter, cheese, cottage cheese, sour cream, yogurt, frozen yogurt, Gelato, sherbet, sorbet, ice cream, or whey protein.

In some embodiments, the category of the food is liquid, and the type of liquid is beverage, edible oil, or other liquid. In some embodiments, the type of liquid is beverage, and the species of beverage is beer, cider, coffee, juice, milk, water, or alcoholic beverage. In some embodiments, the type of liquid is edible oil, and the species of edible oil is coconut oil, corn oil, cottonseed oil, olive oil, palm oil, peanut oil, rapeseed oil, canola oil, safflower oil, sesame oil, soybean oil, sunflower oil, almond oil, beech nut oil, brazil nut oil, cashew oil, hazelnut oil, macadamia oil, mongongo nut oil, pecan oil, pine nut oil, pistachio oil, walnut oil, pumpkin seed oil, grapefruit seed oil, lemon oil, orange oil, gourd oil, butternut squash seed oil, egusi seed oil, watermelon seed oil, acai oil, black seed oil, blackcurrant seed oil, borage seed oil, evening primrose oil, flaxseed oil, linseed oil, amaranth oil, apricot oil, apple seed oil, argan oil, avocado oil, babassu oil, ben oil, borneo tallow nut oil, cape chestnut oil, carob pod oil, cocoa butter, cocklebur oil, cohune oil, coriander seed oil, date seed oil, dika oil, false flax oil, grape seed oil, kapok seed oil, kenaf seed oil, lallemantia oil, mafura oil, marula oil, meadowfoam seed oil, mustard oil, niger seed oil, nutmeg butter, okra seed oil, papaya seed oil, perilla seed oil, persimmon seed oil, pili nut oil, pomegranate seed oil, poppyseed oil, pracaxi oil, prune kernel oil, quinoa oil, ramtil oil, rice bran oil, royle oil, sapote oil, seje oil, shea butter, taramira oil, tea seed oil, thistle oil, tigernut oil, tobacco seed oil, tomato seed oil, or wheat germ oil. In some embodiments, the type of liquid is other liquid, and the species of other liquid is syrup, honey, or molasses.

In some embodiments, a food is selected from the group consisting of apple, blueberry, banana, grape, and tomato. In some embodiments a grape is a green seedless grape. In some embodiments, a food is selected from the group consisting of apple, avocado, banana, blueberry, green grape, red grape, strawberry, spinach, and tomato.

### Analytes

In some embodiments, a critical composition profile (“CCP”) for a food includes analyte data (or “nutrient data”) for one or more analytes (or “nutrients”). In some embodiments, a food's CCP includes one or more analytes selected from the group consisting of water, sugar, vitamin C, malic acid, citric acid, antioxidants, and soluble solids. In some embodiments, a food's CCP includes four or more analytes selected from the group consisting at least one sugar, at least one acid, at least one vitamin, at least one mineral, at least one fat, at least one starch, at least one fiber, at least one carotenoid, at least one flavonoid, at least one protein, moisture content, alcohol content, and/or gluten.

Some non-limiting examples of analyte types include sugars, acids, vitamins, biotins, minerals, fats, starches, fibers, carotenoids, and flavonoids. In some embodiments, the sugar type of analytes may include (without limitation) sucrose, glucose, fructose, and/or total sugar content. In some embodiments, sugar is fructose, fructose or a mixture thereof. In some embodiments, the acid type of analytes may include (without limitation) malic acid, citric acid, folic acid, oxalic acid, lactic acid, ascorbic acid, fatty acid, and/or total acid content. In some embodiments, the vitamin type of analytes may include vitamin A, one or more biotins, vitamin C, vitamin D, vitamin E, and/or vitamin K. In some embodiments, the biotin type of analytes may include vitamin B-1, vitamin B-2, vitamin B-3, vitamin B-5, vitamin B-6, vitamin B-7, vitamin B-9, and/or vitamin B12.

In some embodiments, the mineral type of analytes may include calcium, chloride, fluoride, iron, manganese, magnesium, phosphorous, potassium, sodium, and/or zinc. In some embodiments, the fat type of analytes may include saturated fat, trans fat, unsaturated fat, and/or total fat. In some embodiments, the starch type of analytes may include potato starch, wheat starch, tapioca starch, corn starch, rice starch, and/or total starch. In some embodiments, the fiber type of analytes may include soluble fiber, insoluble fiber, and/or total fiber. In some embodiments, the fiber type of analytes may include cellulose, hemicellulose, inulin oligofructose, lignin, mucilage, beta-glucan, pectin, gum, polydextrose polyol, psyllium, resistant starch, and/or wheat dextrin. In some embodiments, the carotenoid type of analytes may include (without limitation) alpha-carotene, beta-carotene and/or lycopene. In some embodiments, the flavonoid type of analytes may include at least one anthoxanthin, at least one flavone, at least one flavanol, at least one flavanone, at least one flavanonol, at least one flavan, at least one anthocyanidin, and/or at least one anthocyanin.

### Reference Values for Analytes

In this subsection, reference is made to Tables 1-9. Each of the tables corresponds to a food and lists analytes that may be included in some embodiments of the food's critical composition profile. The tables also list reference values (“amounts” or “levels”) for the indicated analytes, according to some embodiments. The reference values used for the selected analytes in some embodiments of the systems and methods described herein may be approximately equal to the reference values listed in Tables 1-9. The reference values may be derived from nutritional standards reported by the U.S. Department of Agriculture (“USDA”) for the food, or from a survey of other literature (“Lit”). The notation “NR” is used to identify analytes for which USDA reference values are not reported.

In some cases, Tables 1-9 indicate “total” reference levels for analyte classes. The “total” reference levels for an analyte class may be reported as an equivalent level of the most prevalent compound of that class. In some cases, analyte levels reported as “total” reference levels have been determined via analysis of the individual analytes and summation of the individual analyte levels.

Referring to Table, 1, in some embodiments, one or more (e.g., all) the analyte(s) included in the critical composition profile (CCP) of an apple may be selected from the group consisting of water, vitamin C, glucose, fructose, and malic acid. In some embodiments, one or more (e.g., all) the analyte(s) included in the CCP of an apple are selected from the group consisting of sucrose, glucose, fructose, total sugar content, malic acid, citric acid, vitamin C, total acid content, and/or moisture content. In some embodiments, one or more (e.g., all) the analyte(s) included in the CCP of an apple are selected from those analytes listed in Table 1. In some embodiments, the reference levels of one or more analytes of an apple are selected from (or approximately equal to) those listed in Table 1.

TABLE 1

| | | Sugar, | Sugar, | Sugar, | Acid, | Anthocyanin, | Anti-oxidant | Acid, |
|---|---|---|---|---|---|---|---|---|
| | Moisture | Glucose | Fructose | Sucrose | Ascorbic | Total | Mmol | Malic |
| | g/100 g | g/100 g | g/100 g | g/100 g | mg/100 g | mg/100 g | Trolox/100 g | g/100 g |
| USDA Ave. | 85.56 ± 0.24 | 2.43 ± 0.03 | 5.9 ± 0.05 | 2.07 ± 0.04 | 4.6 ± 0.4 | 1.6 ± 0.4 | NR | NR |
| Lit Low | | | 3.84 | | 0.38 | | 3.275 | 0.193 |
| Lit High | | | 8.01 | | 26.68 | | 3.275 | 1.738 |
| Lit Ave. | | | 5.69 ± 0.84 | | 9.58 | | 3.275 ± 0.249 | 0.847 ± 0.281 |

In some embodiments, one or more (e.g., all) the analyte(s) included in the CCP of an avocado are selected from those analytes listed in Table 2. In some embodiments, the reference levels of one or more analytes of an avocado are selected from (or approximately equal to) those listed in Table 2.

TABLE 2

| | | | | | Fatty Acid, | Fatty Acid, | Anthocyanin, |
|---|---|---|---|---|---|---|---|
| | | | Fatty Acid, | Fatty Acid, | Palmitic | Palmitoleic | Total |
| | Moisture | Lipids (Fat) | Linoleic (18:2) | Oleic (18:1) | (16:0) | (16:1) | mg/g |
| | g/100 g | g/100 g | g/100 g | g/100 g | g/100 g | mg/100 g | (in the peel) |
| USDA Ave. | 73.23 ± 1.89 | 14.66 ± 0.54 | 1.674 ± 0.063 | 9.066 ± 0.474 | 2.075 ± 0.104 | 0.0698 ± 0. | NR |
| Lit Low | 68.6 | 8 | | | | | 0.23 ± 0. |
| Lit High | 78.36 | 33 | | | | | |
| Lit Average | 72.4 | 17 | | | | | |

In some embodiments, one or more (e.g., all) the analyte(s) included in the CCP of a banana are selected from the group consisting of water, potassium, magnesium, vitamin C, and total sugar. In some embodiments, one or more (e.g., all) the analyte(s) included in the CCP of a banana are selected from the group consisting of potassium, magnesium, sucrose, glucose, fructose, total sugar content, malic acid, citric acid, vitamin C, total acid content, and/or moisture content. In some embodiments, one or more (e.g., all) the analyte(s) included in the CCP of a banana are selected from those analytes listed in Table 3. In some embodiments, the reference levels of one or more analytes of a banana are selected from (or approximately equal to) those listed in Table 3.

TABLE 3

| | | Sugar, | Sugar, | Sugar, | Acid, | Acid, | Acid, |
|---|---|---|---|---|---|---|---|
| | Moisture | Glucose | Fructose | Sucrose | Citric | Malic | Ascorbic |
| | g/100 g | g/100 g | g/100 g | g/100 g | g/100 g | g/100 g | mg/100 g |
| USDA Ave. | 74.91 ± 0.28 | 4.98 ± 0.8 | 4.85 ± 0.65 | 2.39 ± 0.54 | NR | NR | 8.7 ± 0.4 |
| Lit Low | 71.9 | 0.20 | 0.10 | 1 | 0.14 | 0.19 | 2 |
| Lit High | 77.5 | 4.20 | 3.20 | 11.2 | 0.23 | 0.365 | 18.7 |
| Lit Average | 74.7 | 2.38 | 1.68 | 7.8 | 0.18 | 0.26 | 10 |

In some embodiments, one or more (e.g., all) the analyte(s) included in the CCP of a blueberry are selected from the group consisting of glucose, fructose, total sugar content, citric acid, vitamin C, total acid content, moisture, and/or anthocyanins. In some embodiments, one or more (e.g., all) the analyte(s) included in the CCP of a blueberry are selected from those analytes listed in Table 4. In some embodiments, the reference level(s) of one or more analytes of a blueberry are selected from (or approximately equal to) those listed in Table 4.

TABLE 4

| | | Sugar, | Sugar, | Anthocyanin, | Acid, |
|---|---|---|---|---|---|
| | Moisture | Glucose | Fructose | Total | Ascorbic |
| | g/100 g | g/100 g | g/100 g | g/100 g | mg/100 g |
| USDA Ave. | 84.21 ± | 4.88 ± | 4.97 ± | 163.26 ± | 9.7 ± |
| | 0.672 | 0.275 | 0.276 | 0.89 | 0.89 |
| Lit Low | | | | | |
| Lit High | | | | | |
| Lit Ave. | | | | | |

In some embodiments, one or more (e.g., all) the analytes included in the CCP of a grape are selected from the group consisting of flavanols, potassium, calcium, malic acid, citric acid, vitamin C, total acid content, and/or moisture content. In some embodiments, one or more (e.g., all) the analyte(s) of a green grape are selected from the group consisting of flavanols, potassium, calcium, malic acid, citric acid, vitamin C, total acid content, and/or moisture content. In some embodiments, one or more (e.g., all) the analyte(s) included in the CCP of a green grape are selected from those analytes listed in Table 5. In some embodiments, the reference levels of one or more analytes of a green grape are selected from (or approximately equal to) those listed in Table 5.

TABLE 5

| | | Sugar, | Sugar, | Acid, | Acid, |
|---|---|---|---|---|---|
| | Moisture | Glucose | Fructose | Tartaric | Malic |
| | g/100 g | g/100 g | g/100 g | g/100 g | g/100 g |
| USDA Ave. | 80.54 ± | 7.2 ± | 8.13 ± | NR | NR |
| | 0.41 | 0.09 | 0.21 | | |
| Lit Low | 82 | 6.60 | 7.30 | 0.4 | 0.15 |
| Lit High | 82 | 12 54 | 15.91 | 0.76 | 0.66 |
| Lit Average | 82 | 9.27 | 10.90 | 0.5619 | 0.3162 |

In some embodiments, one or more (e.g., all) the analyte(s) included in the CCP of a red grape are selected from those analytes listed in Table 6. In some embodiments, the reference levels of one or more analytes of a red grape are selected from (or approximately equal to) those listed in Table 6.

TABLE 6

| | | Sugar, | Sugar, | Anthocyanin, | Acid, | Acid, |
|---|---|---|---|---|---|---|
| | Moisture | Glucose | Fructose | Total | Tartaric | Malic |
| | g/100 g | g/100 g | g/100 g | mg/100 g | g/100 g | g/100 g |
| USDA Ave. | 80.54 ± 0.41 | 7.2 ± 0.09 | 8.13 ± 0.21 | NR | NR | NR |
| Lit Low | 82 | 6.60 | 7.30 | 6.3 | 0.48 | 0.22 |
| Lit High | 82 | 12.54 | 15.91 | 39.7 | 0.6 | 0.33 |
| Lit Average | 82 | 9.27 | 10.90 | 21.7 | 0.5433 | 0.2760 |

In some embodiments, one or more (e.g., all) the analyte(s) included in the CCP of strawberry are selected from the group consisting of glucose, fructose, total sugar content, vitamin C, anthocyanins, citric acid, total acid content, soluble solids, antioxidants, and/or moisture content. In some embodiments, one or more (e.g., all) the analyte(s) included in the CCP of a strawberry are selected from those analytes listed in Table 7. In some embodiments, the reference levels of one or more analytes of a strawberry are selected from (or approximately equal to) those listed in Table 7.

TABLE 7

| | | Sugar, | Sugar, | Acid, | Anthocyanin, | Anti-oxidant | Acid, | Acid, |
|---|---|---|---|---|---|---|---|---|
| | Moisture | Glucose | Fructose | Ascorbic | Total | Mmol | Citric | Malic |
| | g/100 g | g/100 g | g/100 g | mg/100 g | mg/100 g | Trolox/100 g | g/100 g | g/100 g |
| USDA Ave. | 90.95 ± 0.21 | 7.2 ± 0.09 | 2.44 ± 0.19 | 58.8 ± 2.4 | 27.1 ± 2.4 | NR | NR | NR |
| Lit Low | 92.5 | 1.89 | 2.14 | 35 | 7.6 | 0.402 | 0.596 | 0.00 |
| Lit High | 92.5 | 4.52 | 4.14 | 131.9 | 56.4 | 2.060 | 1.434 | 0.69 |
| Lit Average | 92.5 ± 0.8 | 1.59 | 1.93 | 78 | 32.0 | 1.231 | 1.014 | 0.17 |

In some embodiments, one or more (e.g., all) the analyte(s) included in the CCP of spinach are selected from those analytes listed in Table 8. In some embodiments, reference levels of one or more analytes of spinach are selected from (or approximately equal to) those listed in Table 8.

TABLE 8

| | | Acid, | Anti-oxidant | Acid, | Carotenoid, | Carotenoid, |
|---|---|---|---|---|---|---|
| | Moisture | Ascorbic | Mmol | Oxalic | total | Lutein |
| | g/100 g | mg/100 g | Trolox/100 g | g/100 g | μg/100 g | μg/100 g |
| USDA Ave. | 91.4 | 28.1 ± 4.1 | NR | NR | NR | 12198 ± 1930 |
| Lit Low | 92.17 | 9 | 1.778 | 0.3 | 32000 | 33500 |
| Lit High | 94.7 | 60.6 | 2.183 | 1.3 | 32000 | 53000 |
| Lit Average | 93.335 | 30 | 1.981 | 0.6 | 32000 | 46433 ± 8592 |

In some embodiments, one or more (e.g., all) the analyte(s) included in the CCP of a tomato are selected from the group consisting of water, lycopene, vitamin C, potassium and beta-carotene. In some embodiments, one or more (e.g., all) the analyte(s) of a tomato are selected from lycopene, potassium, beta-carotene, malic acid, citric acid, vitamin C, total acid content, and/or moisture content. In some embodiments, one or more (e.g., all) the analyte(s) included in the CCP of a tomato are selected from those analytes listed in Table 9. In some embodiments, reference levels of one or more analytes of a tomato are selected from (or approximately equal to) those listed in Table 9.

TABLE 9

| | | | | | Anti- | | | | |
|---|---|---|---|---|---|---|---|---|---|
| | | | | | oxidant | | | | |
| | | Sugar, | Sugar, | Acid, | Mmol | Acid, | Acid, | Carotenoid, | Carotenoid, |
| | Moisture | Glucose | Fructose | Ascorbic | Trolox/ | Citric | Malic | total | Lycopene |
| | g/100 g | g/100 g | g/100 g | mg/100 g | 100 g | g/100 g | g/100 g | μg/100 g | μg/100 g |
| USDA | 94.52 ± 0.13 | 1.25 ± 0.13 | 1.37 ± 0.07 | 13.7 ± 0.8 | NR | NR | NR | NR | 2573 ± 054 |
| Ave. | | | | | | | | | |
| Lit | 93.8 | 2.81 | 1.35 | 15 | 1.321 | 0.2681 | 0.0514 | 6274 | 2300 |
| Low | | | | | | | | | |
| Lit | 94.6 | 3.01 | 1.75 | 22 | 4.524 | 0.5402 | 0.1201 | 9832 | 8474 |
| High | | | | | | | | | |
| Lit | 94.2 ± 0.6 | 2.91 ± 0.06 | 1.65 ± 0.15 | 17 ± 2 | 3.082 ± 1.632 | 0.3926 ± 0.0917 | 0.0929 ± 0.0241 | 7575 | 5831 |
| Average | | | | | | | | | |

### Spectrometric Data Collection

In some embodiments, the following data collection protocol can be used to perform the spectroscopic analysis of the samples in step 108 of the method 100:
Step 1. Load food-specific parameters
   a. Parameters (e.g., integration time, number of scans to average, etc.) can be entered by the user or selected from a menu of parameters corresponding to each food.
   b. Collection parameters may be food type dependent (e.g., different parameters for fruit and vegetables, meats, cheeses, oils, etc.).
   c. Instrument ranges may be tied to the type of food.
      i. E.g., because fruits and vegetables are high in water, a spectroscopic range from about 350 nm to about 1700 nm may be preferred for scanning fruits and vegetables.
      ii. E.g., for cheese or oils, a spectroscopic range from about 350 nm to about 2200 nm may be preferred.
Step 2. Prompt probe to perform a “dark” scan and a background scan.
   a. All chosen ranges are collected and stored using the parameters defined.
   b. A quality check of the background data is done by comparing the background data to an average background from each spectrometer for the specified parameters.
Step 3. Prompt probe to perform a scan on a known reference sample.
   a. A reference sample is analyzed by the probe.
   b. Once collected the quality check of the reference data is done by comparing the spectral data for the reference sample to an average from each spectrometer for the specified parameters.
Step 4. Prompt probe to scan a sample.
   a. Load ID and metadata for sample. In some cases, the sample's ID and metadata can be selected from a dropdown menu linked to a lookup table containing metadata. In some cases, the sample's ID and metadata can be loaded by scanning a bar code.
   b. Obtain spectrum of sample.
   c. Check for quality. If quality of spectrum is poor, prompt for repeat of data. Some components used to determine quality of a scan may include: level of response, signal-to-noise ratio, and/or whether the sample is in focus.
   d. Store each range in a separate directory with the sample name as the ID and the range as the appended suffix.
   e. If multiple samples are run from the same metadata set they are assigned a sample number/index to differentiate the sample runs for later use.
   f. Store all data to allow for input into subsequent model prediction.
Step 5. Store data in appropriate format for archiving.
Step 6. Prompt for repeat of dark, background, and reference scans.
   a. Periodically (e.g., every 0.5 hours), or
   b. If quality check shows 2 back to back poor-quality scans.
Step 7. At end of set of runs, verify that all data is saved to disk to make sure no data is lost.

### Generating Measurement Models

In some embodiments, measurement models for a food can be used to accurately estimate the amounts of specific analytes present in samples of the food based on spectral data generated from spectroscopic scans of the samples. Such measurement models can be generated using any suitable technique. In some embodiments, measurements models for a food are machine learning models. In some embodiments, such measurement models are trained and validated using analyte measurements and spectral data for a set of samples of the food.

In some embodiments, a method for generating a measurement model is provided. The measurement model may be suitable for accurately estimating the amount of an analyte present in samples of a food based on spectral data generated from one or more spectroscopic scans of the samples. In some embodiments, the method for generating the measurement model includes obtaining sample spectroscopic data indicating spectroscopic characteristics of a plurality of samples of the food; obtaining sample analytical data indicating measured amounts of the analyte present in the samples; and training the measurement model to estimate an amount of the analyte in a specimen of the food based at least in part on specimen spectroscopic data indicating spectroscopic characteristics of the specimen, wherein values of one or more independent variables of the measurement model are derived from the spectroscopic characteristics of the samples, wherein values of one or more dependent variables of the measurement model are derived from the measured amounts of the analyte present in the samples, and wherein training the measurement model comprises fitting the measurement model to the values of the independent variables and the values of the dependent variables.

In some embodiments, the analyte is a contaminant, an adulterant, and/or a substance indicative of food spoilage.

In some embodiments, obtaining the sample spectroscopic data comprises receiving the sample spectroscopic data via a network or loading the sample spectroscopic data from a computer-readable storage medium. In some embodiments, obtaining the sample spectroscopic data comprises performing one or more spectroscopic scans of the samples. In some embodiments, performing the spectroscopic scans comprises scanning the samples using absorption spectroscopy, emission spectroscopy, elastic scattering spectroscopy, impedance spectroscopy, inelastic scattering spectroscopy, and/or coherent spectroscopy. In some embodiments, scanning the samples using absorption spectroscopy comprises scanning the samples using infrared spectroscopy, ultraviolet spectroscopy, and/or visible spectroscopy. In some embodiments, scanning the samples using emission spectroscopy comprises scanning the samples using flame emission spectroscopy, fluorescence spectroscopy, energy-dispersive X-ray spectroscopy, and/or X-ray fluorescence. In some embodiments, scanning the samples using impedance spectroscopy comprises scanning the samples using electrochemical impedance spectroscopy (EIS). In some embodiments, scanning the samples using coherent spectroscopy comprises scanning the samples using coherent Stokes Raman spectroscopy, coherent anti-Stokes Raman spectroscopy, Raman spectroscopy, nuclear magnetic resonance (NMR) spectroscopy, and/or ultrafast laser spectroscopy.

In some embodiments, the sample spectroscopic data indicate measurements of electromagnetic waves associated with the samples at X-ray wavelengths, ultraviolet (UV) wavelengths, visible wavelengths, infra-red wavelengths, thermal infrared wavelengths, and/or microwave wavelengths. In some embodiments, the sample spectroscopic data are obtained using one or more laboratory-quality spectroscopes. In some embodiments, the sample spectroscopic data are obtained using one or more field-quality spectroscopes.

In some embodiments, the specimen spectroscopic data is received via a network or loading the specimen spectroscopic data from a computer-readable storage medium. In some embodiments, the specimen spectroscopic data is obtained by performing one or more spectroscopic scans of the specimen. In some embodiments, the specimen spectroscopic data indicate measurements of electromagnetic waves associated with the specimen at X-ray wavelengths, ultraviolet (UV) wavelengths, visible wavelengths, infra-red wavelengths, thermal infrared wavelengths, and/or microwave wavelengths. In some embodiments, the specimen spectroscopic data are obtained using one or more laboratory-quality spectroscopes. In some embodiments, the specimen spectroscopic data are obtained using one or more field-quality spectroscopes.

In some embodiments, the measurement model is a linear multivariate regression model, a non-linear multivariate regression model, or a blend of two or more of the foregoing. In some embodiments, the linear multivariate regression model is a multiple linear regression model, a principal component regression model, a partial least square regression model, or a support vector regression model. In some embodiments, the non-linear multivariate regression model is a kernel partial least square regression model, a kernel support vector regression model, or a neural network-based regression model. In some embodiments, a kernel function of the kernel partial least square regression model is a polynomial function, a spline, or a non-linear transformation.

In some embodiments, training the measurement model further comprises pre-processing training data prior to fitting the model, wherein the training data comprise the sample spectroscopic data. In some embodiments, pre-processing the training data comprises performing dimension reduction, differentiation, normalization, smoothing, averaging, scaling, and/or augmentation on the training data.

In some embodiments, the sample spectroscopic data are divided into a plurality of sub-bands, wherein each sub-band corresponds to one or more ranges of wavelengths and includes a portion of the sample spectroscopic data indicating measurements of the electromagnetic waves associated with the samples at wavelengths within the corresponding range of wavelengths. In some embodiments, the sub-bands are overlapping. In some embodiments, the sub-bands are non-overlapping.

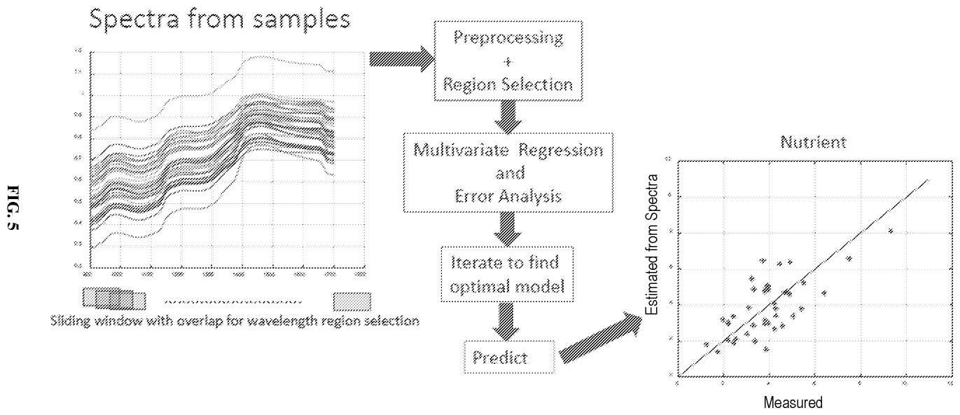

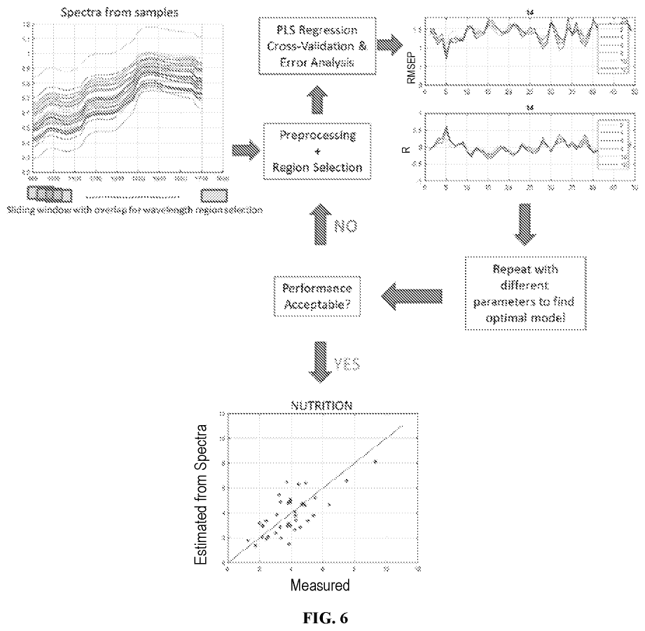

In some embodiments, fitting the measurement model comprises: for each sub-band in the plurality of sub-bands, fitting a candidate measurement model to the portion of the sample spectroscopic data corresponding to the selected sub-band; evaluating performance of a plurality of potential measurement models selected from a group comprising the candidate measurement models and one or more blended measurement models, wherein each blended measurement model comprises a blend of two or more of the candidate measurement models; and based on the performance of the potential measurement models, selecting a particular potential measurement model as the fitted measurement model. See FIGS. 5 and 6.

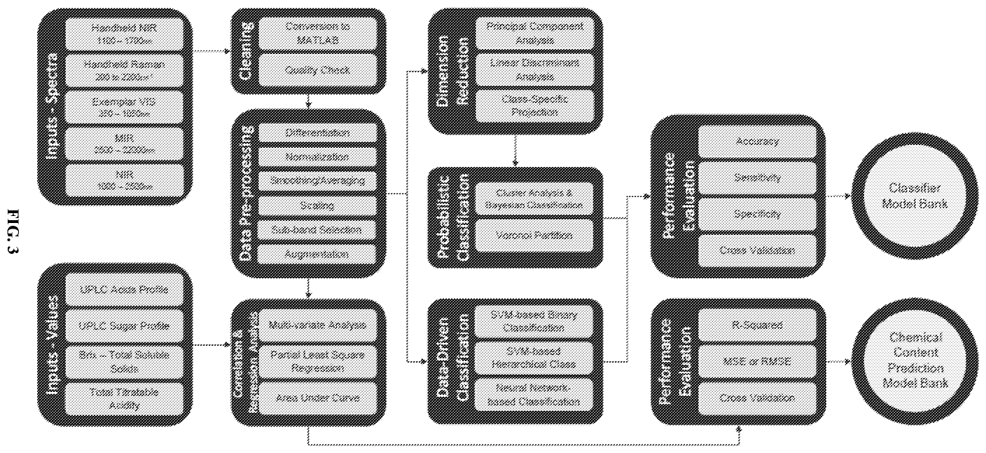

As just one example, a measurement model may be developed using one or more of the model development techniques illustrated in FIG. 3.

### Detecting Contaminants

In some embodiments, an analyte may be a contaminant, an adulterant, and/or a substance indicative of food spoilage.

In some embodiments, a contaminant detection method comprises identifying one or more potential contaminants and/or adulterants of the sample. For each of the potential contaminants and/or adulterants, the method further includes: obtaining a respective model of a relationship between amounts of the potential contaminant or adulterant in the food and spectroscopic characteristics of the food within one or more ranges of wavelengths corresponding to the model; and using the model to determine an amount of the potential contaminant or adulterant in the sample based on a respective portion of the spectroscopic data indicating spectroscopic characteristics of the sample at wavelengths within the range of wavelengths corresponding to the model.

In some embodiments, identifying the potential contaminants or adulterants of the sample comprises accessing a data record for the identified food, wherein the data record indicates the potential contaminants and/or adulterants corresponding to the identified food. In some embodiments, the method further includes determining whether the amount of a potential contaminant or adulterant exceeds a threshold amount, and if so, recommending a recall of a set of samples including the sample.

In some embodiments, the identified food is tea and the potential contaminant is sawdust, or the identified food is milk formula and the potential contaminant is melamine.

In some embodiments, identifying the potential contaminants and/or adulterants of the sample comprises receiving data indicating an advertised characteristic of the sample; and accessing a data record corresponding to the advertised characteristic, wherein the data record indicates potential contaminants inconsistent with the advertised characteristic.

In some embodiments, data indicating an advertised characteristic of sample is derived from a label associated with the sample. In some embodiments, an advertised characteristic of the sample is a vegetarian characteristic and a potential contaminant is meat.

In some embodiments, a potential contaminant or adulterant is one or more analytes selected from the group consisting of an added sugar, a synthetic fragrance, a chemical additive, a pathogen, a pesticide, and a diluent. In some embodiments, a chemical additive is a flavor enhancer, a dye, and/or an extender. In some embodiments, a chemical additive is StabilEase, glutamic acid, monosodium glutamate (MSG), monopotassium glutamate, calcium diglutamate, monoammonium glutamate, magnesium diglutamate, guanylic acid, disodium guanylate, sodium guanylate, dipotassium guanylate, calcium guanylate, inosinic acid, disodium inosinate, dipotassium inosinate, calcium inosinate, calcium 5′-ribonucleotides, disodium 5′-ribonucleotides, maltol, ethyl maltol, glycine, glycine sodium salt, L-Leucine, 2,4-dithiapentane, cellulose, soy protein, or vegetable protein.

### Classification of Food Samples

In some embodiments, classification models suitable for accurately identifying a food “class” to which a food specimen belongs based on the specimen's spectral data (e.g., based on attributes of the specimen's identity and/or context derived from the specimen's spectral data) may be generated. In some embodiments, a classification model may be generated based on correlations between (1) spectroscopic data indicating spectral characteristics of samples of different foods and (2) labels identifying the food classes to which the samples belong. Such labels may, for example, be assigned to the samples by humans.

In some embodiments, a classification model is trained using a machine learning algorithm, with the spectroscopic sample data and the class label data for a suitable set of samples serving as the training and validation data. In some embodiments, a method for generating a classification model for classifying a specimen of a food includes: obtaining sample spectroscopic data indicating spectroscopic characteristics of a plurality of samples of a plurality of foods; obtaining food classification data indicating the food classes of the samples; and training the classification model to classify specimens of the foods based at least in part on specimen spectroscopic data indicating spectroscopic characteristics of the specimens, wherein values of one or more independent variables of the model are derived from the spectroscopic characteristics of the samples, wherein values of one or more dependent variables of the model are derived from food classification data for the samples, and wherein training the model comprises fitting the classification model to the values of the independent variables and the values of the dependent variables.

In some embodiments, generating the classification model further comprises pre-processing training data prior to fitting the model, wherein the training data comprise the sample spectroscopic data. In some embodiments, pre-processing training data comprises performing dimension reduction, differentiation, normalization, smoothing, averaging, scaling, and/or augmentation on the training data on the training data. In some embodiments, performing dimension reduction comprises performing principal component analysis, linear discriminant analysis, and/or class-specific projection on the training data.

A classification model may include, for example, one or more machine learning classifiers. In some embodiments, a classification model may accurately identify the food classes to which food samples belong based on spectral data generated from spectroscopic scans of the samples. In some cases, suitable spectral data of a sample can be obtained using a brief scan with a field spectrometer, and the classification model can be used to identify the sample's food class based on the spectral data in real time (e.g., within 10 seconds or less).

In some embodiments, a classification model includes a plurality of classifiers. In some embodiments, the classification model comprises (1) one or more graph-based classifiers, (2) one or more support vector machine (SVM) based classifiers, (3) one or more neural network-based classifiers, and/or one or more blended classifiers obtained by blending of two or more of the foregoing types of classifiers.

In some embodiments, a classification model is operable to hierarchically classify a specimen to one or more different levels of specificity (e.g., according to category, type, species, and/or subspecies). In some embodiments, the classification model is operable to classify a food sample as being a sample of an apple, avocado, banana, blueberry, green grape, red grape, strawberry, spinach, or tomato.

In some embodiments, a classification model may be used to identify a sample's food class, and then a measurement model specific to the determined food class of the sample may be selected based on the classification, which may facilitate faster and/or more accurate estimation of the amounts of specific analytes in the specimen. If the system is unable to resolve certain attributes of the specimen's identity or context with high confidence, the system may select a measurement model that is more general to a broader food class that includes the specimen but is less specific to the specimen.

In some embodiments, classification models may be used to determine whether food samples are authentic. In some embodiments, determining whether a sample is authentic comprises: receiving data indicating an advertised identity of the food; determining whether the classification of the sample matches the advertised identity of the food, and if so, determining that the sample is authentic; and otherwise, determining that the sample is not authentic.

### Generating Predictive Models

### Generating Predictive Models for Inferring Properties of Food Samples

In some embodiments, predictive models for samples of a food are generated based on correlations between (1) spectroscopic data indicating spectral characteristics of a set of samples of the food and (2) property data indicating values of one or more properties of the samples. In some embodiments, such predictive models may accurately infer certain properties of food samples (e.g., the class of food to which the sample belongs; the region where the sample was produced; the amounts of specific analytes contained in the sample; etc.) based on spectral data generated from spectrometric scans of the samples and/or based on portions of property data. In some cases, suitable spectral data of a sample can be obtained using a brief scan with a field spectrometer, and the predictive model can be used to infer properties of the sample based on the spectral data (and/or portions of property data) in real time (e.g., within 10 seconds or less).

Some examples of properties of food samples may include analytical properties, identity properties, and contextual properties. Analytical properties of the samples may include the amounts of one or more analytes in the samples. Identity properties of a sample may include a “class” of the sample, for example, a category, sub-category (“type”), sub-sub-category (“species”), and/or sub-sub-sub-category (“subspecies”) of foods to which the sample belongs. Contextual properties of a sample may include production data (e.g., data identifying a producer of the sample and/or the production practices used to produce the sample), supply data (e.g., data identifying a supplier of the sample and/or supply practices used to load, unload, distribute, store, process, and/or package the sample), retail data (e.g., data identifying a retailer of the sample or retails practices used to unload, store, process, package, and/or present the sample), and/or scoring data (e.g., data indicating scores of aspects of the sample at different stages in the production and distribution chain).

In some embodiments, a method for generating a predictive model for inferring one or more properties of a specimen of a food includes: obtaining sample spectroscopic data indicating spectroscopic characteristics of a plurality of samples of the food; obtaining sample property data indicating values of one or more properties of the samples; and training a predictive model to determine values of the one or more properties of a specimen of the food based at least in part on specimen spectroscopic data indicating spectroscopic characteristics of the specimen, wherein values of one or more independent variables of the predictive model are derived from the spectroscopic characteristics of the samples, wherein values of one or more dependent variables of the predictive model are derived from the values of the properties of the samples, and wherein training the predictive model comprises fitting the predictive model to the values of the independent variables and the values of the dependent variables.

In some embodiments, the sample property data include analytical data, identity data, and/or contextual data. In some embodiments, obtaining the sample property data comprises receiving the sample property data via a network or loading the sample spectroscopic data from a computer-readable storage medium.

In some embodiments, the sample property data include the identity data, and the identity data for each sample indicate one or more attributes of an identity of the sample. In some embodiments, the attributes of the identity of the sample include a category of the sample, a type of the sample, a species of the sample, a subspecies of the sample, a cultivar of the sample, a producer of a germplasm from which the sample was grown, a location in which the germplasm was produced, and/or a production batch number of a batch of germplasms including the germplasm.

In some embodiments, the sample property data include the contextual data, and the contextual data for each sample include production data, supply data, retail data, and/or scoring data for the sample. In some embodiments, the production data indicate an identity of a producer of the sample and/or a location in which the sample was produced. In some embodiments, the production data include planting data, weather data, and/or farming data associated with the sample. In some embodiments, the supply data indicate an identity of a supplier of the sample. In some embodiments, the supply data include loading data, transportation data, off-loading data, processing data, and/or storage data for the sample. In some embodiments, the loading data indicate a date and/or time at which the sample was loaded onto transportation equipment of a supplier, and/or weather conditions during loading of the sample onto the transportation equipment. In some embodiments, the transportation data indicate: a date and/or time at which transportation of the sample began, a duration of the transportation, a type of shipping container occupied by the sample during the transportation, a type of transportation vehicles used for the transportation, and/or one or more measurements of one or more aspects of an environment of the sample during the transportation. In some embodiments, the off-loading data indicate: a date and/or time at which the sample was off-loaded from a transportation vehicle of a supplier, weather conditions during off-loading of the sample, handling of the sample performed during the off-loading of the sample, a location of the sample after the off-loading of the sample, and/or one or more measurements of one or more aspects of an environment of the sample during the off-loading. In some embodiments, the processing data indicate one or more attributes of one or more types of processing applied to the sample. In some embodiments, the storage data indicate one or more measurements of one or more aspects of an environment of the sample during storage.

In some embodiments, the retail data indicate an identity of a retailer of the sample, one or more measurements of one or more aspects of an environment of the sample during packaging, a type of packaging in which the sample is packaged, one or more measurements of one or more aspects of an environment of the sample during storage, a duration of the storage, one or more measurements of one or more aspects of an environment of the sample during presentation to potential purchasers, and/or a duration of the presentation.

In some embodiments, the scoring data indicate one or more scores representing an aspect of the sample at one or more respective stages of production and/or distribution of the sample. In some embodiments, the aspect of the sample is an overall quality of the sample, a ripeness of the sample, an extent of spoilage of the sample, a characteristic of a texture of the sample, or a freshness of the sample.

In some embodiments, the sample property data include the analytical data, and the analytical data for each sample indicate respective amounts of one or more analytes in the sample. In some embodiments, the analytes include a contaminant, an adulterant, and/or a substance indicative of food spoilage.

In some embodiments, obtaining the sample spectroscopic data comprises receiving the sample spectroscopic data via a network or loading the sample spectroscopic data from a computer-readable storage medium. In some embodiments, obtaining the sample spectroscopic data comprises performing one or more spectroscopic scans of the samples. In some embodiments, performing the spectroscopic scans comprises scanning the samples using absorption spectroscopy, emission spectroscopy, elastic scattering spectroscopy, impedance spectroscopy, inelastic scattering spectroscopy, and/or coherent spectroscopy.

In some embodiments, the sample spectroscopic data indicate measurements of electromagnetic waves associated with the samples at X-ray wavelengths, ultraviolet (UV) wavelengths, visible wavelengths, infra-red wavelengths, thermal infrared wavelengths, and/or microwave wavelengths. In some embodiments, the sample spectroscopic data are obtained using one or more lab-quality spectroscopes. In some embodiments, the sample spectroscopic data are obtained using one or more field-quality spectroscopes.

In some embodiments, obtaining the specimen spectroscopic data comprises receiving the specimen spectroscopic data via a network or loading the specimen spectroscopic data from a computer-readable storage medium. In some embodiments, obtaining the specimen spectroscopic data comprises performing one or more spectroscopic scans of the specimen. In some embodiments, the specimen spectroscopic data indicate measurements of electromagnetic waves associated with the specimen at X-ray wavelengths, ultraviolet (UV) wavelengths, visible wavelengths, infra-red wavelengths, thermal infrared wavelengths, and/or microwave wavelengths. In some embodiments, the specimen spectroscopic data are obtained using one or more lab-quality spectroscopes. In some embodiments, the specimen spectroscopic data are obtained using one or more field-quality spectroscopes.

The predictive model may have any suitable attributes. In some embodiments, the predictive model is a linear multivariate regression model, a non-linear multivariate regression model, or a blend of two or more of the foregoing. In some embodiments, the linear multivariate regression model is a multiple linear regression model, a principal component regression model, a partial least square regression model, or a support vector regression model. In some embodiments, the non-linear multivariate regression model is a kernel partial least square regression model, a kernel support vector regression model, or a neural network-based regression model. In some embodiments, a kernel function of the kernel partial least square regression model is a polynomial function, a spline, or a non-linear transformation.

In some embodiments, training the predictive model further comprises pre-processing training data prior to fitting the predictive model, wherein the training data comprise the sample spectroscopic data and/or the sample property data. In some embodiments, pre-processing the training data comprises performing dimension reduction, differentiation, normalization, smoothing, averaging, scaling, and/or augmentation on the training data.

The method may further comprise dividing the sample spectroscopic data into a plurality of sub-bands, wherein each sub-band corresponds to one or more ranges of wavelengths and includes a portion of the sample spectroscopic data indicating measurements of the electromagnetic waves associated with the samples at wavelengths within the corresponding range of wavelengths. In some embodiments, the sub-bands are overlapping. In some embodiments, the sub-bands are non-overlapping. In some embodiments, fitting the predictive model comprises: for each sub-band in the plurality of sub-bands, fitting a candidate predictive model to the portion of the sample spectroscopic data corresponding to the selected sub-band and the property data; evaluating performance of a plurality of potential predictive models selected from a group comprising the candidate predictive models and one or more blended predictive models, wherein each blended predictive model comprises a blend of two or more of the candidate predictive models; and based on the performance of the potential predictive models, selecting a particular potential predictive model as the fitted predictive model.

In some embodiments, the sample property data are first sample property data; the properties of the samples are first properties of the samples; the method further comprises obtaining second sample property data indicating values of one or more second properties of the samples; and the predictive model is trained to determine values of the first properties of the specimen of the food based on the specimen spectroscopic data and on specimen property data indicating values of the one or more second properties of the specimen. In some embodiments, the independent variables of the predictive model are first independent variables; values of one or more second independent variables of the predictive model are derived from the values of the second properties of the samples; and the predictive model is fitted to the values of the first independent variables, the values of the second independent variables, and the values of the dependent variables.

### Generating Predictive Models for Inferring Scores of Food Samples

The food quality assessment system may obtain longitudinal data regarding the changes in individual samples' scores as the samples move through the food production and distribution chain, analyze that data to assess the performance of entities participating in the production and distribution chain and their practices, and recommend or initiate adjustments to the supply chain based on that analysis. In this way, the food quality assessment system can use the longitudinal data to improve the quality of food obtained via the food supply chain by a user of the system and can enable users of the system to exert pressure on producers, suppliers, and retailers to provide higher-quality food. Such longitudinal data have not been available in the past, partly because conventional techniques for measuring the amounts of analytes present in a food sample either cause significant degradation in the sample's quality or cause the sample to be destroyed, thereby making it difficult or impossible to obtain accurate measurements repeatedly as the sample moves through the food supply chain. Enabling the collection of such longitudinal data is an important benefit of some embodiments of the techniques described herein. Furthermore, the longitudinal data and associated contextual data may be used to generate models that can accurately predict the scores for aspects of a specimen based on the specimen's identity and context, even without obtaining spectrographic scans of the specimen.

In some embodiments, a method for generating a predictive model of a score for an aspect of a specimen of a food may include obtaining identity data, contextual data, and scoring data for a plurality of samples of the food, wherein the identity data for each sample indicate one or more values of one or more attributes of an identity of the sample, wherein the scoring data for each sample include two or more scores representing the aspect of the sample at two or more respective stages of production and/or distribution of the sample, wherein the contextual data for each sample include two or more contextual datasets corresponding to the two or more respective stages of production and/or distribution of the sample, and wherein each contextual dataset indicates one or more values of one or more attributes of the corresponding stage of production and/or distribution of the corresponding sample; and training a predictive model to predict the score for the aspect of a specimen of the food at a specified stage in production and/or distribution of the specimen based on the identity data, the scoring data, and the contextual data, wherein the values of the attributes indicated in the identity data and the contextual data are values of independent variables and the scores indicated in the scoring data are values of a dependent variable, and wherein training the predictive model comprises fitting the predictive model to the identity data, the contextual data, and the scoring data.

The scoring data may be obtained using any suitable technique. In some embodiments, obtaining the scoring data comprises generating the two or more scores representing the aspect of each sample of the food at the two or more respective stages of production and/or distribution of the respective sample.

In some embodiments, the two or more scores representing the aspect of the respective sample comprise a first score and a second score, the two or more stages of production and/or distribution of the respective sample comprises a first stage and a second stage, and generating the two or more scores representing the aspect of the respective sample at the two or more respective stages of production and/or distribution of the respective sample comprises: generating the first score representing the aspect of the respective sample while the respective sample is at the first stage of production and/or distribution; and generating the second score representing the aspect of the respective sample while the respective sample is at the second stage of production and/or distribution.

In some embodiments, generating the first score representing the aspect of the respective sample while the respective sample is at the first stage of production and/or distribution includes: identifying one or more analytes associated with the aspect of the sample; while the sample is at the first stage of production and/or distribution, spectroscopically scanning the respective sample, thereby obtaining spectroscopic data indicating spectroscopic characteristics of the sample at a plurality of wavelengths; using one or more predictive models to predict amounts of the identified analytes in the respective sample based on the spectroscopic data; and determining the first score based on (1) the determined amounts of the identified analytes in the sample and (2a) respective target amounts of one or more individual analytes included in the identified analytes and/or (2b) respective target values of one or more analyte expressions, wherein each analyte expression includes a respective combination of at least two of the identified analytes.

Some embodiments of techniques for generating scores representing aspects of respective food samples are described herein. In some embodiments, the spectroscopic data indicating the spectroscopic characteristics of the sample are obtained while the respective sample is at the first stage of production and/or distribution. In some embodiments, the first score is generated without destroying the respective sample. In some embodiments, the first score is generated without physically penetrating the respective sample. In some embodiments, the first score is generated without causing a substantial change in the aspect of the respective sample. In some embodiments, the first score is generated without changing any aspect of the respective sample.

The identity data may have any suitable attributes. In some embodiments, the attributes of the identity data for a particular sample of the food comprise a category of the food, a type of the food, a species of the sample, a cultivar of the sample, a producer of a germplasm from which the sample was grown, a location in which the germplasm was produced, and/or a production batch number of a batch of germplasms including the germplasm. In some embodiments, the germplasm comprises a seed, a plurality of a plant cells, or a part of a plant.

The contextual data may have any suitable attributes. In some embodiments, the stages of production and/or distribution of each sample are selected from a group comprising one or more production stages, one or more supply stages, and/or one or more retail stages. In some embodiments, the production stage for a particular sample includes one or more growing stages, a harvest stage, one or more processing stages, and/or one or more storage stages. In some embodiments, the contextual data corresponding to the production stage for the particular sample include producer data and production location data. In some embodiments, the producer data indicate an identity of a producer of the particular sample and/or an identity of a farm where the particular sample was produced, and/or the production location data indicate a country in which the particular sample was produced, a region in which the particular sample was produced, a state in which the particular sample was produced, and/or a field in which the particular sample was produced.

In some embodiments, the contextual data corresponding to a particular growing stage for the particular sample include planting data, weather data, and/or farming data associated with the particular sample. In some embodiments, the planting data indicate a date of planting a germplasm that produced the particular sample; the weather data indicate weather conditions in a location where the particular sample was produced during a time period preceding the planting of the germplasm and/or during a time period preceding harvesting of the particular sample; and/or the farming data indicate (1) attributes of one or more farming interventions applied to a location in which the particular sample was produced prior to the planting of the germplasm and/or during a time period preceding harvesting of the particular sample, (2) attributes of one or more foods provided to an animal that produced the particular sample, and/or (3) availability of shade in the location in which the particular sample was produced. In some embodiments, the farming interventions include application of one or more fertilizers, application of one or more pre-harvest conditioners, irrigation practices, pest control practices, and/or freeze mitigation practices. In some embodiments, the contextual data corresponding to a particular harvest stage for the particular sample indicate a harvest date on which the particular sample was harvested and/or a harvest technique used to harvest the particular sample.

In some embodiments, the supply stage for a particular sample includes one or more loading stages, one or more transportation stages, one or more off-loading stages, one or more processing stages, and/or one or more storage stages. In some embodiments, the contextual data corresponding to the supply stage indicate an identity of a supplier of the particular sample. In some embodiments, the contextual data corresponding to a particular loading stage for the particular sample indicate a date and/or time at which the particular sample was loaded onto transportation equipment of a supplier, and/or weather conditions during loading of the particular sample onto the transportation equipment.

In some embodiments, the contextual data corresponding to a particular transportation stage for the particular sample indicate: a date and/or time at which transportation of the particular sample began, a duration of the transportation, a type of shipping container occupied by the particular sample during the transportation, a type of transportation vehicles used for the transportation, and/or one or more measurements of one or more aspects of an environment of the particular sample during the transportation. In some embodiments, the aspects of the environment include temperature, humidity, vibration, shock, atmospheric pressure, light exposure, lux intensity, and/or off-gassing. In some embodiments, the measurements of at least one aspect of the environment indicate a frequency with which the aspect of the environment was measured, a rate of change in a measured value of the aspect of the environment, and/or a duration of a time period during which a measured value of the aspect of the environment remained stable.

In some embodiments, the contextual data corresponding to a particular off-loading stage for the particular sample indicate: a date and/or time at which the particular sample was off-loaded from a transportation vehicle of a supplier, weather conditions during off-loading of the particular sample, handling of the particular sample performed during the off-loading of the particular sample, a location of the particular sample after the off-loading of the particular sample, and/or one or more measurements of one or more aspects of an environment of the sample during the off-loading. In some embodiments, the handling of the particular sample includes breakbulk processing, crossdocking, and/or repackaging. In some embodiments, the location of the particular sample after the off-loading of the particular sample is selected from a group of locations comprising one or more warehouses and one or more transportation vehicles.

In some embodiments, the contextual data corresponding to a particular processing stage for the particular sample indicate one or more attributes of one or more types of processing applied to the particular sample. In some embodiments, the types of processing includes one or more types of gas processing, one or more types of antibiotic treatment, one or more types of sterilization treatment, one or more slaughtering techniques, and/or one or more packaging processes. In some embodiments, the contextual data corresponding to a particular storage stage for the particular sample indicate one or more measurements of one or more aspects of an environment of the particular sample during storage. In some embodiments, the aspects of the environment include temperature, humidity, vibration, shock, atmospheric pressure, light exposure, lux intensity, and/or off-gassing.

In some embodiments, the retail stage for a particular sample includes one or more packaging stages, one or more storage stages, and/or one or more presentation stages. In some embodiments, the contextual data corresponding to the retail stage indicate an identity of a retailer of the particular sample. In some embodiments, the contextual data corresponding to a particular packaging stage for the particular sample indicate: one or more measurements of one or more aspects of an environment of the particular sample during packaging, and/or a type of packaging in which the particular sample is packaged.

In some embodiments, the contextual data corresponding to a particular storage stage for the particular sample indicate one or more measurements of one or more aspects of an environment of the particular sample during storage. In some embodiments, the aspects of the environment include temperature, humidity, vibration, shock, atmospheric pressure, light exposure, lux intensity, and/or off-gassing.

In some embodiments, the contextual data corresponding to a particular presentation stage for the particular indicate: one or more measurements of one or more aspects of an environment of the particular sample during presentation to potential purchasers, and/or a duration of the presentation. In some embodiments, the production stages precede the supply stages, and wherein the supply stages precede the retail stages.

The aspect of the specimen represented by the score may be an overall quality of the specimen, a ripeness of the specimen, an extent of spoilage of the specimen, an attribute of a texture of the specimen, a freshness of the specimen, or a shelf life of the specimen.

### Using Predictive Models

Predictive models generated in accordance with the above-described techniques may be used for any suitable purpose(s), including but not limited to predicting the current or future score for an aspect of a specimen, recommending or initiating an action with respect to a sample based on its inferred score; recommending or initiating an adjustment to the supply chain based on inferred scores of samples; various forms of supply chain analysis and management; and/or determining whether food samples are authentic.

### Predicting the Current or Future Score for an Aspect of a Specimen

In some embodiments, a predictive model may be used to predict one or more scores for an aspect of a particular specimen of a food at one or more stages in production and/or distribution of the particular specimen. In some embodiments, the scores are predicted by applying the predictive model to input data comprising identity data for the particular specimen and contextual data for the particular specimen.

In some embodiments, the scores for the aspect of the particular specimen include a first score representing a predicted current state of the aspect of the particular specimen; the identity data for the particular specimen indicate one or more values of one or more attributes of an identity of the particular specimen; the contextual data for the particular specimen include one or more first contextual datasets corresponding to one or more respective prior stages of production and/or distribution of the particular specimen, wherein each first contextual dataset indicates one or more values of one or more attributes of the corresponding stage of production and/or distribution of the particular specimen; and the first score is predicted by applying the predictive model to input data comprising the identity data for the particular specimen and the first contextual datasets.

In some embodiments, the input data further include prior scoring data for the particular specimen, and the prior scoring data for the particular specimen include one or more prior scores representing the aspect of the sample at the one or more respective prior stages of production and/or distribution of the particular specimen. In some embodiments, the one or more prior scores are determined based on one or more respective prior spectroscopic datasets each indicating spectroscopic characteristics of the particular specimen while the particular specimen was at the corresponding prior stage of production and/or distribution. In some embodiments, at least one of the prior scores is predicted. In some embodiments, the identity data for the particular specimen and the first contextual datasets are obtained from a distributed ledger associated with a supply chain of the particular specimen.

In some embodiments, the scores for the aspect of the particular specimen include a second score representing a predicted future state of the aspect of the particular specimen; the identity data for the particular specimen indicate one or more values of one or more attributes of an identity of the particular specimen; the contextual data for the particular specimen include one or more first contextual datasets and one or more second contextual datasets, the first contextual datasets corresponding to one or more respective prior and/or present stages of production and/or distribution of the particular specimen, the second contextual datasets corresponding to one or more respective present and/or future stages of production and/or distribution of the particular specimen, wherein each first or second contextual dataset indicates one or more values of one or more attributes of the corresponding stage of production and/or distribution of the particular specimen; and the second score is predicted by applying the predictive model to input data comprising the identity data for the particular specimen and the first and second contextual datasets.

In some embodiments, the input data further include prior scoring data for the particular specimen, and the prior scoring data for the particular specimen include one or more prior scores representing the aspect of the sample at the one or more respective prior and/or present stages of production and/or distribution of the particular specimen. In some embodiments, the one or more prior scores are determined based on one or more respective prior spectroscopic datasets each indicating spectroscopic characteristics of the particular specimen while the particular specimen was at the corresponding prior stage of production and/or distribution. In some embodiments, at least one of the prior scores is predicted.

In some embodiments, a particular second contextual dataset corresponds to a particular future stage of production and/or distribution of the particular specimen, and at least a subset of the attribute values in the particular second contextual dataset are estimated based on an identity of a producer of the particular specimen, an identity of a supplier of the particular specimen, and/or an identity of a retailer of the particular specimen. In some embodiments, the identity data for the particular specimen and the first contextual datasets are obtained from a distributed ledger associated with a supply chain of the particular specimen.

### Recommending or Initiating an Action With Respect to a Sample

In some embodiments, an action with respect to a sample may be recommended or initiated based on the sample's inferred score. In some embodiments, the scores for the particular specimen include a score for an overall quality of the particular specimen. In some embodiments, a food quality assessment system makes a determination to accept or reject delivery of a shipment of specimens of the food including the particular specimen based, at least in part, on the overall quality score for the particular specimen. In some embodiments, delivery of the shipment of specimens is accepted or rejected (e.g., by the system) in accordance with the determination.

In some embodiments, the system assigns the particular specimen to a grouping based, at least in part, on the overall quality score, wherein the grouping is one of a plurality of groupings. In some embodiments, a food-handling machine places the particular specimen in a container of specimens corresponding to the assigned grouping, wherein the container is one of a plurality of containers corresponding to the plurality of groupings. In some embodiments, a sale price or a purchase price for the particular specimen or a set of samples including the particular specimen is determined based, at least in part, on the overall quality score for the particular specimen.

### Recommending or Initiating an Adjustment to the Supply Chain

In some embodiments, an adjustment to the food supply chain may be recommended or initiated based on the inferred score(s) of one or more samples.

In some embodiments, the system makes a determination to adjust one or more agricultural characteristics of a farm where the particular specimen was produced based, at least in part, on the overall quality score for the particular specimen. In some embodiments, the one or more agricultural characteristics of the farm where the particular specimen was grown are adjusted in accordance with the determination. In some embodiments, the agricultural characteristics include (1) planting practices, (2) harvesting practices, (3) irrigation practices, (4) fertilization practices, (5) tillage practices, (6) practices associated with application of fungicides, pesticides, antimicrobials, nucleic acids, and/or biologicals, and/or (7) variety of crop grown.

In some embodiments, the system makes a determination to adjust operating parameters of a food storage facility or container based, at least in part, on the overall quality score for the particular specimen. In some embodiments, the operating parameters of the food storage facility or container are adjusted (e.g., by the system) in accordance with the determination. In some embodiments, the particular specimen is or was in the food storage facility or container. In some embodiments, the operating parameters of the food storage facility or container include temperature levels, humidity levels, and/or light levels.

### Supply Chain Analysis and Management (First Embodiment)

In some embodiments, a method for food supply-chain analysis and/or management includes, for each of a plurality of potential values of a parameter of a supply chain for a food: obtaining scoring data indicating a plurality of scores representing an aspect of a respective plurality of samples of the food at a specified stage of production or distribution, wherein the samples correspond to the respective potential value of the parameter; and determining a value of a statistical measure of the scores of the samples corresponding to the respective potential value of the parameter. The method further includes providing, via a user interface, information associated with the determined values of the statistical measure; and based on the determined values of the statistical measure of the respective scores of the samples corresponding to the respective potential values of the parameter, adjusting the supply chain for the food.

Location-specific analysis and/or adjustment of the supply chain for a food may be performed using any suitable techniques. In some embodiments, the parameter of the supply chain for the food is a location in which at least a portion of the food in the supply chain is produced, and the potential values of the parameter correspond to respective locations in which specimens of the food are produced.

In some embodiments, adjusting the supply chain for the food comprises adding a plurality of the specimens produced in a particular location to the supply chain or removing a plurality of the specimens produced in a particular location from the supply chain. In some embodiments, the particular location is represented by a particular potential value of the parameter of the supply chain, the value of the statistical measure of the scores associated with the particular location is a particular value of the statistical measure, and the supply chain is adjusted based on the particular value of the statistical measure satisfying one or more criteria.

In some embodiments, the criteria are satisfied if the particular value of the statistical measure is less than a specified threshold value, greater than a specified threshold value, within a range of values between two specified values, less than the other values of the statistical measure associated with the other respective values of the parameter, and/or greater than the other values of the statistical measure associated with the other respective values of the parameter.

In some embodiments, the locations in which specimens of the food are produced include a first location and a second location, the value of the statistical measure of the scores associated with the first location is superior to the value of the statistical measure of the scores associated with the second location, and adjusting the supply chain for the food comprises changing one or more food production practices at the second location to more closely conform to one or more respective food production practices at the first location.

In some embodiments, providing the information associated with the values of the statistical measure comprises ranking the locations based on the respective values of the statistical measure associated with the locations; and presenting the locations in rank order via the user interface.

Producer-specific analysis and/or adjustment of the supply chain for a food may be performed using any suitable techniques. In some embodiments, the parameter of the supply chain for the food is a producer of at least a portion of the food in the supply chain, and the potential values of the parameter correspond to respective producers of specimens of the food.

In some embodiments, adjusting the supply chain for the food comprises adding a plurality of the specimens produced by a particular producer to the supply chain or removing a plurality of the specimens produced by a particular producer from the supply chain. In some embodiments, the particular producer is represented by a particular potential value of the parameter of the supply chain, the value of the statistical measure of the scores associated with the particular producer is a particular value of the statistical measure, and the supply chain is adjusted based on the particular value of the statistical measure satisfying one or more criteria.

In some embodiments, the criteria are satisfied if the particular value of the statistical measure is less than a specified threshold value, greater than a specified threshold value, within a range of values between two specified values, less than the other values of the statistical measure associated with the other respective values of the parameter, and/or greater than the other values of the statistical measure associated with the other respective values of the parameter.

In some embodiments, the producers of specimens of the food include a first producer and a second producer, the value of the statistical measure of the scores associated with the first producer is superior to the value of the statistical measure of the scores associated with the second producer, and adjusting the supply chain for the food comprises changing one or more food production practices of the second producer to more closely conform to one or more respective food production practices of the first producer.

In some embodiments, providing the information associated with the values of the statistical measure comprises ranking the producers based on the respective values of the statistical measure associated with the producers; and presenting the producers in rank order via the user interface.

Supplier-specific analysis and/or adjustment of the supply chain for a food may be performed using any suitable techniques. In some embodiments, the parameter of the supply chain for the food is a supplier of at least a portion of the food in the supply chain, and the potential values of the parameter correspond to respective suppliers of specimens of the food.

In some embodiments, adjusting the supply chain for the food comprises adding a plurality of the specimens supplied by a particular supplier to the supply chain or removing a plurality of the specimens supplied by a particular supplier from the supply chain. In some embodiments, the particular supplier is represented by a particular potential value of the parameter of the supply chain, the value of the statistical measure of the scores associated with the particular supplier is a particular value of the statistical measure, and the supply chain is adjusted based on the particular value of the statistical measure satisfying one or more criteria.

In some embodiments, the criteria are satisfied if the particular value of the statistical measure is less than a specified threshold value, greater than a specified threshold value, within a range of values between two specified values, less than the other values of the statistical measure associated with the other respective values of the parameter, and/or greater than the other values of the statistical measure associated with the other respective values of the parameter.

In some embodiments, the suppliers of specimens of the food include a first supplier and a second supplier, the value of the statistical measure of the scores associated with the first supplier is superior to the value of the statistical measure of the scores associated with the second supplier, and adjusting the supply chain for the food comprises changing one or more operational practices of the second supplier to more closely conform to one or more respective operational practices of the first supplier.

In some embodiments, providing the information associated with the values of the statistical measure comprises ranking the suppliers based on the respective values of the statistical measure associated with the suppliers; and presenting the suppliers in rank order via the user interface.

Retailer-specific analysis and/or adjustment of the supply chain for a food may be performed using any suitable techniques. In some embodiments, the parameter of the supply chain for the food is a retailer of at least a portion of the food in the supply chain, and the potential values of the parameter correspond to respective retailers of specimens of the food.

In some embodiments, wherein adjusting the supply chain for the food comprises adding a plurality of the specimens offered by a particular retailer to the supply chain or removing a plurality of the specimens offered by a particular retailer from the supply chain. In some embodiments, the particular retailer is represented by a particular potential value of the parameter of the supply chain, the value of the statistical measure of the scores associated with the particular retailer is a particular value of the statistical measure, and the supply chain is adjusted based on the particular value of the statistical measure satisfying one or more criteria.

In some embodiments, the criteria are satisfied if the particular value of the statistical measure is less than a specified threshold value, greater than a specified threshold value, within a range of values between two specified values, less than the other values of the statistical measure associated with the other respective values of the parameter, and/or greater than the other values of the statistical measure associated with the other respective values of the parameter.

In some embodiments, the retailers of specimens of the food include a first retailer and a second retailer, the value of the statistical measure of the scores associated with the first retailer is superior to the value of the statistical measure of the scores associated with the second retailer, and adjusting the supply chain for the food comprises changing one or more operational practices of the second retailer to more closely conform to one or more respective operational practices of the first retailer.

In some embodiments, providing the information associated with the values of the statistical measure comprises ranking the retailers based on the respective values of the statistical measure associated with the retailers; and presenting the retailers in rank order via the user interface.

Statistical measures of the scores for a set of samples may be determined using any suitable techniques. In some embodiments, the statistical measure of a particular plurality of scores comprises a measure of a central tendency of the particular scores, a measure of a statistical dispersion of the particular scores, a measure of a shape of a distribution of the particular scores, and/or a measure of a statistical dependence of the particular scores on one or more parameters of the supply chain.

In some embodiments, the measure of the central tendency of the particular scores comprises a mean, median, mode, and/or interquartile mean of the particular scores. In some embodiments, the measure of the statistical dispersion of the particular scores comprises a standard deviation, variance, range, interquartile range, and/or coefficient of variation of the particular scores. In some embodiments, the measure of the shape of the distribution comprises a skewness and/or kurtosis of the distribution of the particular scores. In some embodiments, the measure of the statistical dependence of the particular scores on the one or more parameters of the supply chain is a correlation coefficient. In some embodiments, the correlation coefficient is a Pearson correlation coefficient or a Spearman's rank correlation coefficient.

The scoring data may be obtained using any suitable techniques. In some embodiments, the scoring data indicating a particular plurality of scores representing the aspect of a particular plurality of samples of the food at the specified stage of production or distribution are first scoring data, and obtaining the first scoring data comprises generating the particular plurality of scores representing the aspect of the particular plurality of samples of the food at the specified stage of production or distribution.

In some embodiments, generating the particular plurality of scores comprises, for each of the particular samples, generating the particular score representing the aspect of the particular sample while the particular sample is at the specified stage of production or distribution.

In some embodiments, generating the particular score representing the aspect of the particular sample while the particular sample is at the specified stage of production or distribution includes: identifying one or more analytes associated with the aspect of the particular sample; while the particular sample is at the specified stage of production or distribution, spectroscopically scanning the particular sample, thereby obtaining spectroscopic data indicating spectroscopic characteristics of the particular sample at a plurality of wavelengths; using one or more predictive models to predict amounts of the identified analytes in the particular sample based on the spectroscopic data; and determining the particular score based on (1) the determined amounts of the identified analytes in the particular sample and (2a) respective target amounts of one or more individual analytes included in the identified analytes and/or (2b) respective target values of one or more analyte expressions, wherein each analyte expression includes a respective combination of at least two of the identified analytes.

In some embodiments, the particular score representing the aspect of the particular sample of the food is generated using measurement models and a scoring engine as described herein, wherein the spectroscopic data indicating the spectroscopic characteristics of the particular sample are obtained while the particular sample is at the specified stage of production or distribution.

In some embodiments, the particular score is generated without destroying the particular sample. In some embodiments, the particular score is generated without physically penetrating the particular sample. In some embodiments, the particular score is generated without causing a substantial change in the aspect of the particular sample.

### Supply Chain Analysis and Management (Second Embodiment)

In some embodiments, another method for food supply-chain analysis and/or management includes, for each of a plurality of suppliers of a food: obtaining first scoring data indicating a plurality of first scores representing an aspect of a respective plurality of samples of the food at a first stage of distribution in which the respective supplier receives the respective samples from a producer or a different supplier, determining a first value of a statistical measure of the first scores corresponding to the respective supplier, obtaining second scoring data indicating a plurality of second scores representing the aspect of the respective plurality of samples at a second stage of distribution in which the respective supplier delivers the respective samples to a retailer, a consumer, or a different supplier, determining a second value of a statistical measure of the second scores corresponding to the respective supplier, and determining a relationship between the first score and the second score for the respective supplier. The method may further include providing, via a user interface, information associated with the relationships between the first and second scores for the respective suppliers; and based on the relationships between the first and second scores for the respective suppliers, adjusting the supply chain for the food.

Metrics that indicate how well a supplier preserves food quality may be determined using any suitable techniques. In some embodiments, determining the relationship between the first score and the second score for a particular supplier comprises determining a difference between the first score and the second score for the particular supplier and/or a time rate of score decay from a first time at which the particular supplier receives the respective samples to a second time at which the particular supplier delivers the respective samples.

In some embodiments, the difference is an absolute difference or a percentage difference. In some embodiments, the time rate of score decay is a time rate of absolute change in score from the first time to the second time or a time rate of percentage change in score from the first time to the second time. In some embodiments, determining the relationship between the first score and the second score for the particular supplier further comprises normalizing the difference between the first score and the second score based on a freshness of the respective samples at the first time at which the particular supplier receives the respective samples, and/or normalizing the time rate of score decay based on said freshness of the respective samples.

The supply chain may be adjusted using any suitable techniques. In some embodiments, adjusting the supply chain for the food comprises adding a plurality of specimens supplied by a particular supplier to the supply chain or removing a plurality of specimens supplied by a particular supplier from the supply chain. In some embodiments, the relationship between the first and second scores for the particular supplier is represented by a particular value, and the supply chain is adjusted based on the particular value satisfying one or more criteria.

In some embodiments, the criteria are satisfied if the particular value representing the relationship between the first and second scores for the particular supplier is less than a specified threshold value, greater than a specified threshold value, within a range of values between two specified values, less than other values representing the respective relationships between the first and second scores for the other respective suppliers, and/or greater than the other values representing the respective relationships between the first and second scores for the other respective suppliers.

In some embodiments, the suppliers of the food include a first supplier and a second supplier, a first value representing the relationship between the first and second scores for the first supplier is superior to a second value representing the relationship between the first and second scores for the second supplier, and adjusting the supply chain for the food comprises changing one or more operational practices of the second supplier to more closely conform to one or more respective operational practices of the first supplier.

In some embodiments, providing the information associated with the relationships between the first and second scores for the respective suppliers comprises: ranking the suppliers based on respective values representing the respective relationships between the first and second scores for the respective suppliers; and presenting the suppliers in rank order via the user interface.

The scoring data may be obtained using any suitable techniques. In some embodiments, obtaining the first scoring data comprises, for each of the suppliers: generating the respective plurality of first scores representing the aspect of the respective plurality of samples of the food at the first stage of distribution.

In some embodiments, generating a particular plurality of first scores representing the aspect of a particular plurality of samples of the food at the first stage of distribution comprises, for each of the particular samples: generating the particular first score representing the aspect of the particular sample while the particular sample is at the first stage of distribution. In some embodiments, generating the particular first score representing the aspect of the particular sample while the particular sample is at the first stage of distribution includes: identifying one or more analytes associated with the aspect of the particular sample; while the particular sample is at the first stage of distribution, spectroscopically scanning the particular sample, thereby obtaining spectroscopic data indicating spectroscopic characteristics of the particular sample at a plurality of wavelengths; using one or more predictive models to predict amounts of the identified analytes in the particular sample based on the spectroscopic data; and determining the particular first score based on (1) the determined amounts of the identified analytes in the particular sample and (2a) respective target amounts of one or more individual analytes included in the identified analytes and/or (2b) respective target values of one or more analyte expressions, wherein each analyte expression includes a respective combination of at least two of the identified analytes.

In some embodiments, the particular first score representing the aspect of the particular sample of the food is generated using measurement models and a scoring engine as described herein, wherein the spectroscopic data indicating the spectroscopic characteristics of the particular sample are obtained while the particular sample is at the specified first stage of distribution.

In some embodiments, the particular first score is generated without destroying the particular sample. In some embodiments, the particular first score is generated without physically penetrating the particular sample. In some embodiments, the particular first score is generated without causing a substantial change in the aspect of the particular sample.

In some embodiments, obtaining the second scoring data comprises, for each of the suppliers: generating the respective plurality of second scores representing the aspect of the respective plurality of samples of the food at the second stage of distribution. In some embodiments, generating a particular plurality of second scores representing the aspect of a particular plurality of samples of the food at the second stage of distribution comprises, for each of the particular samples: generating the particular second score representing the aspect of the particular sample while the particular sample is at the second stage of distribution.

In some embodiments, generating the particular second score representing the aspect of the particular sample while the particular sample is at the second stage of distribution includes: identifying one or more analytes associated with the aspect of the particular sample; while the particular sample is at the second stage of distribution, spectroscopically scanning the particular sample, thereby obtaining spectroscopic data indicating spectroscopic characteristics of the particular sample at a plurality of wavelengths; using one or more predictive models to predict amounts of the identified analytes in the particular sample based on the spectroscopic data; and determining the particular second score based on (1) the determined amounts of the identified analytes in the particular sample and (2a) respective target amounts of one or more individual analytes included in the identified analytes and/or (2b) respective target values of one or more analyte expressions, wherein each analyte expression includes a respective combination of at least two of the identified analytes.

In some embodiments, the particular second score representing the aspect of the particular sample of the food is generated using measurement models and a scoring engine as described herein, wherein the spectroscopic data indicating the spectroscopic characteristics of the particular sample are obtained while the particular sample is at the specified second stage of distribution.

### Supply Chain Analysis and Management (Third Embodiment)

In some embodiments, another method for food supply-chain analysis and/or management includes, for each of a plurality of dates: obtaining scoring data indicating a plurality of scores representing an aspect of a respective plurality of samples of a food at a specified stage of production or distribution for the food on the respective date, and determining a value of a statistical measure of the scores of the samples of the food associated with the respective date. The method further includes determining a relationship between the statistical measure of the scores and a date parameter; providing, via a user interface, information associated with the relationship between the statistical measure of the scores and the date parameter; and based on the relationship between the statistical measure of the scores and the date parameter, adjusting a supply chain for the food.

Time-dependent patterns in the quality of a food provided by the supply chain may be analyzed using any suitable techniques. Time-dependent patterns in the quality of food produced in particular locations, produced by particular producers, supplied by particular suppliers, and/or offered by particular retailers may be analyzed using any suitable techniques.

In some embodiments, the supply chain for the food is adjusted based on the relationship between the statistical measure of the scores and the date parameter indicating that a value of the statistical measure of the scores was and/or will be less than a threshold value between two particular dates, greater than a threshold value between two particular dates, and/or within a specified range of values between two particular dates.

In some embodiments, determining the relationship between the statistical measure of the scores and the date parameter comprises generating a model of the relationship, wherein the model is a linear regression model, a non-linear regression model, or a time-series predictive model. In some embodiments, the time-series predictive model models periodic variation in the relationship. In some embodiments, the periodic variation includes seasonal variation.

In some embodiments, the samples are produced in a particular location. In some embodiments, adjusting the supply chain for the food comprises adding a plurality of specimens produced in the particular location between two specified dates to the supply chain or removing a plurality of specimens produced in the particular location between two specified dates from the supply chain.

In some embodiments, the samples are produced by a particular producer. In some embodiments, adjusting the supply chain for the food comprises adding a plurality of specimens produced by the particular producer between two specified dates to the supply chain or removing a plurality of specimens produced by the particular producer between two specified dates from the supply chain.

In some embodiments, the samples are supplied by a particular supplier. In some embodiments, adjusting the supply chain for the food comprises adding a plurality of specimens supplied by the particular supplier between two specified dates to the supply chain or removing a plurality of specimens supplied by the particular supplier between two specified dates from the supply chain.

In some embodiments, the samples are offered by a particular retailer. In some embodiments, adjusting the supply chain for the food comprises adding a plurality of specimens offered by the particular retailer between two specified dates to the supply chain or removing a plurality of specimens offered by the particular retailer between two specified dates from the supply chain.

In some embodiments, providing the information associated with the relationship between the statistical measure of the scores and the date parameter comprises: generating a graph in which an independent axis represents the date parameter, a dependent axis represents the statistical measure of the scores, and a plurality of plotted points represent the values of the statistical measure at the respective dates included in the plurality of dates; and presenting the graph via the user interface.

In some embodiments, the specified stage of production or distribution is the same for each date included in the plurality of dates.

The scoring data may be obtained using any suitable techniques. In some embodiments, obtaining the scoring data comprises, for each date included in the plurality of dates: generating the respective plurality of scores representing the aspect of the respective plurality of samples of the food at the specified stage of production or distribution on the respective date.

In some embodiments, generating a particular plurality of scores representing the aspect of a particular plurality of samples of the food at the specified stage of production or distribution on a particular date comprises, for each of the particular samples: generating the particular score representing the aspect of the particular sample while the particular sample is at the specified stage of production or distribution on the particular date.

In some embodiments, generating the particular score representing the aspect of the particular sample while the particular sample is at the specified stage of production or distribution on the particular date includes: identifying one or more analytes associated with the aspect of the particular sample; while the particular sample is at the specified stage of production or distribution on the particular date, spectroscopically scanning the particular sample, thereby obtaining spectroscopic data indicating spectroscopic characteristics of the particular sample at a plurality of wavelengths; using one or more predictive models to predict amounts of the identified analytes in the particular sample based on the spectroscopic data; and determining the particular score based on (1) the determined amounts of the identified analytes in the particular sample and (2a) respective target amounts of one or more individual analytes included in the identified analytes and/or (2b) respective target values of one or more analyte expressions, wherein each analyte expression includes a respective combination of at least two of the identified analytes.

In some embodiments, the particular score representing the aspect of the particular sample of the food is generated using measurement models and a scoring engine as described herein, wherein the spectroscopic data indicating the spectroscopic characteristics of the particular sample are obtained while the particular sample is at the specified stage of production or distribution on the particular date.

In some embodiments, the particular score is generated without destroying the particular sample. In some embodiments, the particular score is generated without physically penetrating the particular sample. In some embodiments, the particular score is generated without causing a substantial change in the aspect of the particular sample.

### Determining Whether Food Samples are Authentic

In some embodiments, predictive models may be used to determine whether food samples are authentic. In some embodiments, determining whether the sample is authentic comprises: receiving data indicating an advertised identity of the food; determining whether the attributes of the identified food match corresponding attributes of food having the advertised identity, and if so, determining that the sample is authentic; and otherwise, determining that the sample is not authentic.

### Improving the Fidelity of Low-Resolution Spectrometric Scans of Food Samples

In some embodiments, the food quality assessment system may use predictive models to improve the fidelity or predictive power of low-resolution spectrographic scans of food samples. In some embodiments, a method for generating a predictive model for improving a fidelity of a low-resolution spectrographic scan of a food specimen may include obtaining high-resolution sample spectroscopic data indicating, for each of a plurality of samples of a food, high-resolution measurements of electromagnetic waves associated with the sample within one or more ranges of wavelengths corresponding to the food, wherein a fidelity of the high-resolution sample spectroscopic data is high; obtaining low-resolution sample spectroscopic data indicating, for each of the samples, low-resolution measurements of electromagnetic waves associated with the sample within the one or more ranges of wavelengths corresponding to the food; and training a predictive model to predict high-fidelity measurements of electromagnetic waves associated with a specimen of the food based on low-resolution measurements of electromagnetic waves associated with the specimen, wherein values of one or more independent variables of the predictive model are derived from the low-resolution sample spectroscopic data, wherein values of one or more dependent variables of the predictive model are derived from the high-resolution sample spectroscopic data, and wherein training the predictive model comprises fitting the model to the values of the independent variables and the values of the dependent variables.

In some embodiments, the predictive model may be used to predict high-fidelity measurements of a specimen's spectroscopic characteristics based on a low-resolution spectroscopic scan of the specimen by: obtaining low-resolution specimen spectroscopic data indicating low-resolution measurements of electromagnetic waves associated with the specimen within the one or more ranges of wavelengths corresponding to the food; and executing the predictive model on the low-resolution measurements associated with the specimen, thereby predicting high-fidelity measurements of the electromagnetic waves associated with the specimen within the one or more ranges of wavelengths corresponding to the food.

### EXAMPLE

### Classification of Apples

Fresh-picked orchard apples were harvested and tested in a laboratory setting within 24 hours of harvest. Each sample contained 8 representative apples.

Orchard apples samples (22 total) consisted of the 3 largest U.S. commercial cultivars:
McIntosh 6 samples NY, 6 samples New England
Red Delicious 4 samples NY, 4 samples New England
Fuji 2 samples NY (early production)

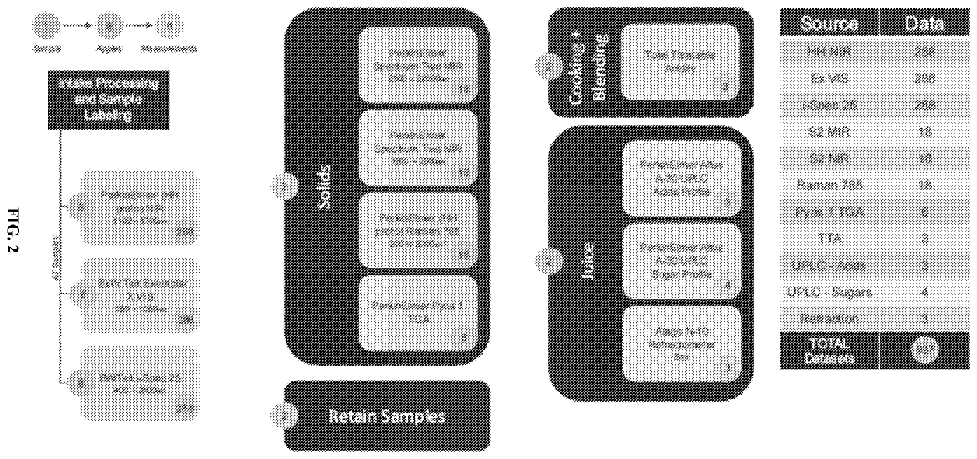

The apple samples were analyzed using various analytical techniques as shown in FIG. 2. The data was processed using the following steps:
5 apples/set (10 apples/orchard) were selected
2nd derivative of the spectral data was used
No other pre-processing techniques used
MATLAB selected for data analytics and modeling
Raw data from lab and handheld spectrometers converted into MATLAB format
Low quality and corrupted data were scrubbed/separated
Handheld-NIR data were selected as first dataset
Binary SVM classifier was trained using Handheld-NIR data for cultivar and orchard level classification
PLS Regression technique is used to estimate averaged brix and titration values from Handheld-NIR data for orchard apples
Repeated random sub-sampling validation—averaged over 50 runs

### Test-1

36 spectra—24 for training/12 for testing (for each apple)

360 spectra—240 for training/120 for testing (for each orchard)

### Test-2

18 spectra—12 for training/6 for testing (for each apple)

180 spectra—120 for training/60 for testing (for each orchard)

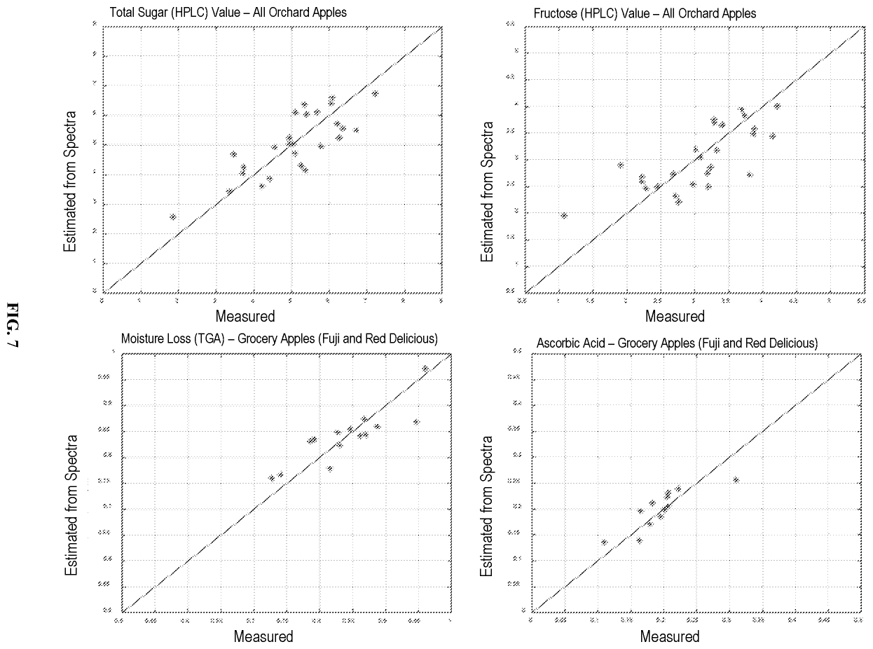

These data were correlated. See FIG. 7

### Results

The following tables show the accuracy rate of a binary classification between cultivars

Fuji vs. McIntosh

| | Wellwood | Macks | North Star | Windy Hill | Middlefield |
|---|---|---|---|---|---|
| | McIntosh | McIntosh | McIntosh | McIntosh | McIntosh |
| Middlefield- | 95.87% | 94.55% | 94.72% | 88.22% | 96.37% |
| Fuji | | | | | |

Red Delicious vs. McIntosh

| | Wellwood | Macks | North Star | Windy Hill | Middlefield |
|---|---|---|---|---|---|
| | McIntosh | McIntosh | McIntosh | McIntosh | McIntosh |
| Riverview | 99.93% | 98.30 | 96.96% | 99.25% | 98.63% |
| Red | | | | | |
| Delicious | | | | | |
| Wellwood | 99.98% | 99.30% | 97.90% | 98.47% | 98.42% |
| Red | | | | | |
| Delicious | | | | | |

Fuji vs. Red Delicious

| | Riverview | Wellwood |
|---|---|---|
| | Red | Red |
| | Delicious | Delicious |
| Middlefield | 97.78% | 94.12% |
| Fuji | | |

With a random mix of apples, the correct cultivar (McIntosh, Red Delicious or Fuji) was identified at an accuracy rate of 92%. Once the cultivar has been identified, the correct orchard (Wellwood Orchards, Riverview Farm) was identified at an accuracy rate of 96%.

In this example, the quantity of samples was very low. In general, a minimum sample quantity of 100 is preferred.

### Scoring Aspects of Food Samples

In some embodiments, the food quality assessment system may (1) use the above-described measurement models to determine the amounts of specific analytes in a sample of a particular food, and (2) assign a score indicating an extent to which an aspect of the sample ‘measures up’ to an objective standard for the quality of samples of that food. In this way, the system can distill a large amount of analytical data into a simple score that entities in the food supply chain (e.g., suppliers, retailers, consumers, etc.) can intuitively understand and easily use to compare different samples and to determine what actions to take with respect to different samples.

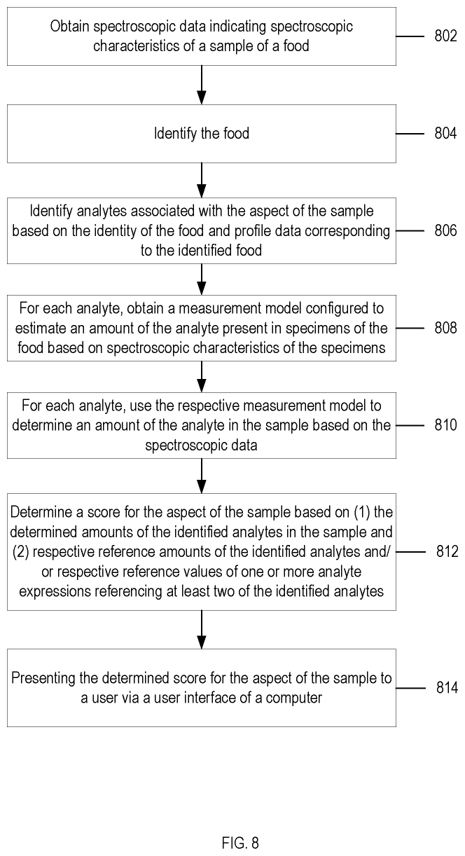

Referring to FIG. 8, a method 800 for scoring an aspect of a sample of a food may include steps of obtaining (802) spectroscopic data indicating spectroscopic characteristics of the sample; identifying (804) the food; and identifying (806) one or more analytes associated with the aspect of the sample based on the identity of the food and profile data corresponding to the identified food. For each of the identified analytes, the method 800 may further include steps of obtaining (808) a respective measurement model configured to estimate an amount of the analyte present in specimens of the food based on spectroscopic characteristics of the specimens; and using (810) the respective measurement model to determine an amount of the analyte in the sample based on the spectroscopic data. The method 800 may further include steps of determining (812) a score for the aspect of the sample based on (1) the determined amounts of the identified analytes in the sample and (2) respective reference amounts of the identified analytes and/or respective reference values of one or more analyte expressions, wherein each analyte expression includes a respective combination of at least two of the identified analytes, and wherein the profile data indicate the reference amounts and/or reference values; and presenting (814) the determined score for the aspect of the sample to a user via a user interface of a computer.

In some embodiments, the reference amount of each individual analyte is determined based at least in part on a standard amount of the individual analyte specified for the food by a regulatory, academic, or industry standard. In some embodiments, the reference value of each analyte expression is determined based at least in part on a standard value of the analyte expression specified for the food by a regulatory, academic, or industry standard.

In some embodiments, the aspect of the sample is a quality of the sample. In some embodiments, the score representing the quality of the sample comprises a combination of (a) one or more individual analyte scores corresponding to the one or more individual analytes and/or (b) one or more analyte expression scores corresponding to the one or more analyte expressions.

In some embodiments, determining the score representing the quality of the sample comprises, for each of the individual analytes, determining a value representing a relationship between the reference amount of the individual analyte and the determined amount of the individual analyte; and determining the individual analyte score corresponding to the individual analyte based on the value representing the relationship between the reference and determined amount of the individual analyte.

In some embodiments, the relationship between the reference and determined amounts of the individual analyte is a ratio of the determined amount to the reference amount, a difference between the determined amount and the reference amount, or a percentage difference between the determined amount and the reference amount. In some embodiments, the individual analyte score corresponding to a particular individual analyte is determined based on a specified function of the value representing the relationship between the reference and determined amounts of the individual analyte. In some embodiments, the specified function includes a linear function, a non-linear function, a parabolic function, an exponential function, and/or a step function.

In some embodiments, determining the score representing the quality of the sample comprises, for each of the analyte expressions: combining the determined amounts of the analytes included in the analyte expression according to the combination associated with the analyte expression, thereby obtaining a determined value of the analyte expression; determining a value representing a relationship between the reference value of the analyte expression and the determined value of the analyte expression; and determining the analyte expression score corresponding to the analyte expression based on the value representing the relationship between the reference and determined values of the analyte expression.

In some embodiments, the relationship between the reference and determined values of the analyte expression is a ratio of the determined amount to the reference amount, a difference between the determined amount and the reference amount, or a percentage difference between the determined amount and the reference amount. In some embodiments, the combination of at least two analytes included in a particular analyte expression comprises a weighted sum of the at least two analytes, a product of the at least two analytes, a ratio of the at least two analytes, or a specified function of the at least two analytes. In some embodiments, the analyte expression score corresponding to a particular analyte expression is determined based on a specified function of the value representing the relationship between the reference and determined values of the analyte expression. In some embodiments, the specified function includes a linear function, a non-linear function, a parabolic function, an exponential function, and/or a step function.

In some embodiments, each of the individual analyte scores is a numeric value within a range specified for the corresponding individual analyte. In some embodiments, each of the analyte expression scores is a numeric value within a range specified for the corresponding analyte expression. In some embodiments, the combination of the individual analyte scores and/or analyte expression scores is a specified function of the individual analyte scores and/or analyte expression scores.

In some embodiments, the specified function of the individual analyte scores and/or analyte expression scores is a weighted linear sum comprising one or more terms, wherein each of the terms comprises a product of (1) a respective term weight and (2) a respective individual analyte score or a respective analyte expression score. In some embodiments, the terms weights are user-adjustable. In some embodiments, the quality score is a numeric value within a specified range. In some embodiments, the quality score is a classification selected from a set of classifications.

In some embodiments, the method further comprises making a determination to accept or reject delivery of a shipment of samples of the food including the sample based, at least in part, on the quality score for the sample. In some embodiments, the method further comprises accepting or rejecting delivery of the shipment of samples in accordance with the determination.

In some embodiments, the method further comprises assigning the sample to a grouping based, at least in part, on the quality score, wherein the grouping is one of a plurality of groupings. In some embodiments, the method further comprises placing the sample in a container of samples corresponding to the assigned grouping, wherein the container is one of a plurality of containers corresponding to the plurality of groupings, and wherein the placing is performed by a food-handling machine. In some embodiments, the food-handling machine is a robot, and obtaining the spectroscopic data comprises performing a spectroscopic scan of the sample at the plurality of wavelengths using a field spectrometer included in a food-handling component of the robot.

In some embodiments, the method further comprises determining a sale price or a purchase price for the sample or a set of samples including the sample based, at least in part, on the quality score for the sample.

In some embodiments, the method further comprises making a determination to adjust one or more agricultural characteristics of a farm where the sample was harvested based, at least in part, on the overall quality score for the sample. In some embodiments, the method further comprises adjusting the one or more agricultural characteristics of the farm where the sample was harvested in accordance with the determination. In some embodiments, the method further comprises the agricultural characteristics include (1) planting practices, (2) harvesting practices, (3) irrigation practices, (4) fertilization practices, (5) tillage practices, (6) practices associated with application of fungicides, pesticides, antimicrobials, nucleic acids, and/or biologicals, and/or (7) variety of crop grown.

In some embodiments, the method further comprises making a determination to adjust operating parameters of a food storage facility or container based, at least in part, on the overall quality score for the sample. In some embodiments, the method further comprises adjusting the operating parameters of the food storage facility or container in accordance with the determination. In some embodiments, the sample is or was in the food storage facility or container. In some embodiments, the operating parameters of the food storage facility or container include temperature levels, humidity levels, and/or light levels.

In some embodiments, the aspect of the sample is a ripeness of the sample.

In some embodiments, the aspect of the sample is an extent of spoilage of the sample. In some embodiments, the identified analytes include bacteria, mold, and/or yeast, glycerol, lactic acid, butyric acid, and/or alcohol.

In some embodiments, the aspect of the sample is a characteristic of a texture of the sample. In some embodiments, the characteristic of the texture of the sample is hardness, crispiness, crunchiness, softness, springiness, or tackiness.

In some embodiments, the aspect of the sample is a freshness of the sample. In some embodiments, the method further comprises determining whether an advertised freshness of the sample is accurate. In some embodiments, determining whether the advertised freshness of the sample is accurate comprises receiving data indicating the advertised freshness of the sample; determining that the advertised freshness of the sample is accurate if the determined freshness matches the advertised freshness or is more fresh than the advertised freshness; and otherwise, determining that the advertised freshness is inaccurate. In some embodiments, the data indicating the advertised freshness of the sample is derived from a label associated with the sample.

In some embodiments, identifying the food comprises receiving user input indicating the identity of the food. In some embodiments, identifying the food comprises receiving data obtained by scanning a label associated with the sample of the food, and the identity of the food is determined based on the received data. In some embodiments, identifying the food comprises classifying the sample based on at least a portion of the spectroscopic data indicating the spectroscopic characteristics of the sample, wherein classifying the sample includes providing at least the portion of the spectroscopic data as input to a classifier; and executing the classifier on the provided input, wherein the classifier provides output indicating a classification of the sample, and wherein the classification indicates the identity of the food. In some embodiments, the identity of the food includes a category of the food, a type of the food, a species of the food, a subspecies of the food, and/or a provenance of the food.

In some embodiments, the method further comprises determining whether the sample is authentic. In some embodiments, determining whether the sample is authentic comprises receiving data indicating an advertised identity of the food; determining whether the attributes of the identified food match corresponding attributes of food having the advertised identity, and if so, determining that the sample is authentic; and otherwise, determining that the sample is not authentic. In some embodiments, determining whether the sample is authentic comprises receiving data indicating an advertised identity of the food; determining whether the classification of the sample matches the advertised identity of the food, and if so, determining that the sample is authentic; and otherwise, determining that the sample is not authentic.

In some embodiments, the identified analytes include four or more analytes selected from a group comprising at least one sugar, at least one acid, at least one vitamin, at least one mineral, at least one fat, at least one starch, at least one fiber, at least one carotenoid, at least one flavonoid, at least one protein, moisture content, alcohol content, and/or gluten.

In some embodiments, the food is an apple and the identified analytes include sucrose, glucose, fructose, malic acid, ascorbic acid, moisture content, anti-oxidants, and/or total anthocyanins.

In some embodiments, the food is an apple, the identified analytes include carotenoids and chlorophyll, and the analyte expressions include a particular analyte expression comprising a ratio between the amounts of carotenoids and chlorophyll in the sample.

In some embodiments, the food is a blueberry and the identified analytes include glucose, fructose, moisture content, and/or total anthocyanins.

In some embodiments, the food is a banana and the identified analytes include sucrose, glucose, fructose, malic acid, citric acid, ascorbic acid, and/or moisture content.

In some embodiments, the food is a green grape and the identified analytes include malic acid, tartaric acid, moisture content, glucose, and/or fructose.

In some embodiments, the food is a red grape and the identified analytes include malic acid, tartaric acid, moisture content, glucose, fructose, and/or total anthocyanins.

In some embodiments, the food is a tomato and the identified analytes include lycopene, malic acid, citric acid, ascorbic acid, moisture content, glucose, fructose, and/or total carotenoids.

In some embodiments, the food is a strawberry and the identified analytes include glucose, fructose, ascorbic acid, total anthocyanins, citric acid, anti-oxidants, and/or moisture content.

In some embodiments, the food is spinach and the identified analytes include moisture content, ascorbic acid, anti-oxidants, oxalic acid, total carotenoids, and Lutein carotenoids.

In some embodiments, obtaining the spectroscopic data comprises performing a spectroscopic scan of the sample at the plurality of wavelengths.

In some embodiments, the spectroscopic scan is performed by a field spectrometer. In some embodiments, the field spectrometer is hand-held, coupled to an automated food-handling device, or coupled to an automated food-distribution device. In some embodiments, obtaining the spectroscopic data comprises receiving the spectroscopic data via a network or loading the spectroscopic data from a computer-readable storage medium.

In some embodiments, each of the measurement models is a linear multivariate regression model, a non-linear multivariate regression model, or a blend of two or more of the foregoing. In some embodiments, the linear multivariate regression model is a multiple linear regression model, a principal component regression model, or a partial least square regression model. In some embodiments, the non-linear multivariate regression model is a kernel partial least square regression model, a support vector machine regression model, or a neural network-based regression model. In some embodiments, a kernel function of the kernel partial least square regression model is a polynomial function, a spline, or a non-linear transformation.

In some embodiments, the scoring engine provides “context layering” functionality, whereby the scoring engine's raw scores can be combined with additional, contextual information to generate contextual scores. For example, a sample's quality score may be combined with the sample's price to generate a quality-price score. In some embodiments, the quality-price score is calculated based on a ratio between a quality score of the sample and a price of the sample.

In some embodiments, determining a score representing the quality of a sample of a food comprises: (1) estimating the amount in the sample of each of the food's critical analytes, e.g., as described herein, (2) generating an individual analyte score for each critical analyte based on the estimated amount of the analyte in the sample and the reference level of the analyte for the food, and (3) combining the individual analyte scores produce a quality score for the sample (i.e. a “sample quality score”). In some embodiments, the individual analyte score for an analyte is determined based on the ratio or difference between the estimated amount of the analyte and the reference level of the analyte. In some embodiments, a sample quality score may be a weighted summation of the analyte scores for a sample. In some embodiments, a sample quality score comprises a weighted summation of analyte scores, wherein each analyte score may not have equal weight within the summation, such that the sample quality score reflects a preference for (or emphasis on) one or more analytes relative to other analyte(s). In some embodiments, a sample quality score comprises an unweighted summation of analyte scores (or a weighted summation in which each analyte score has equal weight); such a sample quality score does not reflect bias for any analyte or group of analytes over any other.

### Context Layering of Sample Quality Scores

Some techniques for calculating sample quality scores have been described. In some embodiments, a quality-price score for a sample may be calculated based on the ratio between the sample's quality score and the sample's purchase price. In some embodiments, quality-price scores are useful in determining which retailers (or producers, or suppliers) offer samples of a food at the lowest price for the best quality. In some embodiments, quality-price scores are useful in determining the efficiency of a retailer in delivering quality samples of a food at the retailer's price point.

In some embodiments, a quality-distance score (or “eco score”) for a sample may be calculated based on the ratio between (1) the sample's quality score and (2) the distance the sample traveled from its point of production to its current location in the supply chain. In some embodiments, quality-distance scores are useful in determining which retailers (or suppliers) offer the best balance between the quality of samples and the distance traveled by the samples. In some embodiments, quality-distance scores are useful in determining the efficiency of a retailer in delivering quality samples of a food from nearby (e.g., “local”) points of production.

### Further Description of Some Embodiments

In some examples, some or all of the processing described above can be carried out on an endpoint device (e.g., a personal computing device, laptop device, tablet device, smartphone device, etc.), on one or more centralized computing devices, or via cloud-based processing by one or more servers. In some examples, some types of processing occur on one device and other types of processing occur on another device. In some examples, some or all of the data described above can be stored on a personal computing device, in data storage hosted on one or more centralized computing devices, or via cloud-based storage. In some examples, some data are stored in one location and other data are stored in another location. In some examples, quantum computing can be used. In some examples, functional programming languages can be used. In some examples, electrical memory, such as flash-based memory, can be used.

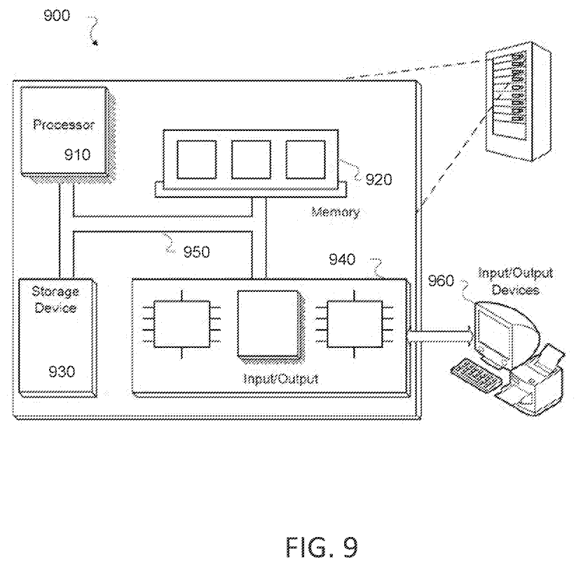

FIG. 9 is a block diagram of an example computer system 900 that may be used in implementing the technology described in this document. General-purpose computers, network appliances, mobile devices, or other electronic systems may also include at least portions of the system 900. The system 900 includes a processor 910, a memory 920, a storage device 930, and an input/output device 940. Each of the components 910, 920, 930, and 940 may be interconnected, for example, using a system bus 950. The processor 910 is capable of processing instructions for execution within the system 900. In some implementations, the processor 910 is a single-threaded processor. In some implementations, the processor 910 is a multi-threaded processor. The processor 910 is capable of processing instructions stored in the memory 920 or on the storage device 930.

The memory 920 stores information within the system 900. In some implementations, the memory 920 is a non-transitory computer-readable medium. In some implementations, the memory 920 is a volatile memory unit. In some implementations, the memory 920 is a non-volatile memory unit.

The storage device 930 is capable of providing mass storage for the system 900. In some implementations, the storage device 930 is a non-transitory computer-readable medium. In various different implementations, the storage device 930 may include, for example, a hard disk device, an optical disk device, a solid-date drive, a flash drive, or some other large capacity storage device. For example, the storage device may store long-term data (e.g., database data, file system data, etc.). The input/output device 940 provides input/output operations for the system 900. In some implementations, the input/output device 940 may include one or more of a network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 960. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 930 may be implemented in a distributed way over a network, for example as a server farm or a set of widely distributed servers, or may be implemented in a single computing device.

Although an example processing system has been described in FIG. 9, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random-access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

The term “approximately,” the term “substantially,” the phrase “approximately equal to,” and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

### Equivalents

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for scoring an aspect of a sample of a food, comprising:
obtaining spectroscopic data indicating spectroscopic characteristics of the sample;
obtaining an identity of the food;
identifying one or more analytes associated with the aspect of the sample based on the identity of the food and profile data corresponding to the identified food;
for each of the identified analytes:
obtaining a respective measurement model configured to estimate an amount of the analyte present in specimens of the food based on spectroscopic characteristics of the specimens; and
using the respective measurement model to determine an amount of the analyte in the sample based on the spectroscopic data;
determining a score for the aspect of the sample based on (1) the determined amounts of the identified analytes in the sample and (2a) respective reference amounts of the identified analytes and/or (2b) respective reference values of one or more analyte expressions, wherein each analyte expression includes a respective combination of at least two of the identified analytes, and wherein the profile data indicate the reference amounts and/or reference values; and
presenting the determined score for the aspect of the sample to a user via a user interface of a computer.

2. The method of claim 1, wherein the aspect of the sample is a quality of the sample.

3. The method of claim 1, wherein the score representing the quality of the sample comprises a combination of (a) one or more individual analyte scores corresponding to the one or more individual analytes and/or (b) one or more analyte expression scores corresponding to the one or more analyte expressions.

4. The method of claim 3, wherein determining the score representing the quality of the sample comprises, for each of the individual analytes:
determining a value representing a relationship between the reference amount of the individual analyte and the determined amount of the individual analyte; and
determining the individual analyte score corresponding to the individual analyte based on the value representing the relationship between the reference and determined amount of the individual analyte.

5. The method of claim 4, wherein the relationship between the reference and determined amounts of the individual analyte is a ratio of the determined amount to the reference amount, a difference between the determined amount and the reference amount, or a percentage difference between the determined amount and the reference amount.

6. The method of claim 4, wherein the individual analyte score corresponding to a particular individual analyte is determined based on a specified function of the value representing the relationship between the reference and determined amounts of the individual analyte.

7. The method of claim 6, wherein the specified function includes a linear function, a non-linear function, a parabolic function, an exponential function, and/or a step function.

8. The method of claim 4, wherein determining the score representing the quality of the sample comprises, for each of the analyte expressions:
combining the determined amounts of the analytes included in the analyte expression according to the combination associated with the analyte expression, thereby obtaining a determined value of the analyte expression;
determining a value representing a relationship between the reference value of the analyte expression and the determined value of the analyte expression; and
determining the analyte expression score corresponding to the analyte expression based on the value representing the relationship between the reference and determined values of the analyte expression.

9. The method of claim 8, wherein the relationship between the reference and determined values of the analyte expression is a ratio of the determined amount to the reference amount, a difference between the determined amount and the reference amount, or a percentage difference between the determined amount and the reference amount.

10. The method of claim 8, wherein the combination of at least two analytes included in a particular analyte expression comprises a weighted sum of the at least two analytes, a product of the at least two analytes, a ratio of the at least two analytes, or a specified function of the at least two analytes.

11. The method of claim 8, wherein the analyte expression score corresponding to a particular analyte expression is determined based on a specified function of the value representing the relationship between the reference and determined values of the analyte expression.

12. The method of claim 11, wherein the specified function includes a linear function, a non-linear function, a parabolic function, an exponential function, and/or a step function.

13. The method of claim 3, wherein each of the individual analyte scores is a numeric value within a range specified for the corresponding individual analyte.

14. The method of claim 3, wherein each of the analyte expression scores is a numeric value within a range specified for the corresponding analyte expression.

15. The method of claim 3, wherein the combination of the individual analyte scores and/or analyte expression scores is a specified function of the individual analyte scores and/or analyte expression scores.

16. The method of claim 15, wherein the specified function of the individual analyte scores and/or analyte expression scores is a weighted linear sum comprising one or more terms, wherein each of the terms comprises a product of (1) a respective term weight and (2) a respective individual analyte score or a respective analyte expression score.

17. The method of claim 16, wherein the terms weights are user-adjustable.

18. The method of claim 16, wherein the quality score is a numeric value within a specified range.

19. The method of claim 18, wherein the quality score is a classification selected from a set of classifications.

20. The method of claim 2, further comprising:
making a determination to accept or reject delivery of a shipment of samples of the food including the sample based, at least in part, on the quality score for the sample.

21. The method of claim 20, further comprising:
accepting or rejecting delivery of the shipment of samples in accordance with the determination.

22. The method of claim 2, further comprising:
assigning the sample to a grouping based, at least in part, on the quality score, wherein the grouping is one of a plurality of groupings.

23. The method of claim 22, further comprising:
placing the sample in a container of samples corresponding to the assigned grouping, wherein the container is one of a plurality of containers corresponding to the plurality of groupings, and wherein the placing is performed by a food-handling machine.

24. The method of claim 23, wherein the food-handling machine is a robot, and wherein obtaining the spectroscopic data comprises performing a spectroscopic scan of the sample at the plurality of wavelengths using a field spectrometer included in a food-handling component of the robot.

25. The method of claim 2, further comprising:
determining a sale price or a purchase price for the sample or a set of samples including the sample based, at least in part, on the quality score for the sample.

26. The method of claim 1, wherein obtaining the identity of the food comprises receiving user input indicating the identity of the food.

27. The method of claim 1, wherein obtaining the identity of the food comprises receiving data obtained by scanning a label associated with the sample of the food, and wherein the identity of the food is determined based on the received data.

28. The method of claim 1, wherein obtaining the identity of the food comprises:
classifying the sample based on at least a portion of the spectroscopic data indicating the spectroscopic characteristics of the sample, wherein classifying the sample includes:
providing at least the portion of the spectroscopic data as input to a classifier; and
executing the classifier on the provided input, wherein the classifier provides output indicating a classification of the sample, and wherein the classification indicates the identity of the food.

29. The method of claim 1, wherein the identified analytes include four or more analytes selected from a group comprising at least one sugar, at least one acid, at least one vitamin, at least one mineral, at least one fat, at least one starch, at least one fiber, at least one carotenoid, at least one flavonoid, at least one protein, moisture content, alcohol content, and/or gluten.

30. The method of claim 1, wherein the food is an apple and the identified analytes include sucrose, glucose, fructose, malic acid, ascorbic acid, moisture content, anti-oxidants, and/or total anthocyanins.

31. The method of claim 1, wherein the food is a blueberry and the identified analytes include glucose, fructose, moisture content, and/or total anthocyanins.

32. The method of claim 1, wherein the food is a banana and the identified analytes include sucrose, glucose, fructose, malic acid, citric acid, ascorbic acid, and/or moisture content.

33. The method of claim 1, wherein the food is a green grape and the identified analytes include malic acid, tartaric acid, moisture content, glucose, and/or fructose.

34. The method of claim 1, wherein the food is a red grape and the identified analytes include malic acid, tartaric acid, moisture content, glucose, fructose, and/or total anthocyanins.

35. The method of claim 1, wherein the food is a tomato and the identified analytes include lycopene, malic acid, citric acid, ascorbic acid, moisture content, glucose, fructose, and/or total carotenoids.

36. The method of claim 1, wherein the food is a strawberry and the identified analytes include glucose, fructose, ascorbic acid, total anthocyanins, citric acid, anti-oxidants, and/or moisture content.

37. The method of claim 1, wherein the food is spinach and the identified analytes include moisture content, ascorbic acid, anti-oxidants, oxalic acid, total carotenoids, and Lutein carotenoids.

38. The method of claim 1, wherein the food is avocado and the identified analytes include moisture content, lipids, linoleic fatty acid, oleic fatty acid, palmitic fatty acid, and/or palmitoleic fatty acid.

39. The method of claim 1, wherein the food is a fruit and the identified analytes include:
moisture content;
at least one sugar selected from the group consisting of glucose and fructose; and
at least one acid selected from the group consisting of ascorbic acid and malic acid.

40. The method of claim 1, wherein obtaining the spectroscopic data comprises performing a spectroscopic scan of the sample at the plurality of wavelengths.

41. The method of claim 40, wherein the spectroscopic scan is performed by a field spectrometer.

42. The method of claim 41, wherein the field spectrometer is hand-held, coupled to an automated food-handling device, or coupled to an automated food-distribution device.

43. The method of claim 1, wherein the measurement model is a linear multivariate regression model, a non-linear multivariate regression model, or a blend of two or more of the foregoing.

44. The method of claim 1, wherein the aspect of the sample is a quality-price index of the sample, wherein the score for the aspect of the sample is a quality-price index score, and wherein the quality-price index score is further based on a price of the sample.

45. The method of claim 44, wherein the quality-price index score is based on a ratio between a price of the sample and a quality score of the sample.

46. A computer system comprising:
one or more processing devices and one or more storage devices storing instructions that are operable, when executed by the processing devices, to cause the processing devices to perform operations including:
obtaining spectroscopic data indicating spectroscopic characteristics of the sample;
obtaining an identity of the food;
identifying one or more analytes associated with the aspect of the sample based on the identity of the food and profile data corresponding to the identified food;
for each of the identified analytes:
obtaining a respective measurement model configured to estimate an amount of the analyte present in specimens of the food based on spectroscopic characteristics of the specimens; and
using the respective measurement model to determine an amount of the analyte in the sample based on the spectroscopic data;
determining a score for the aspect of the sample based on (1) the determined amounts of the identified analytes in the sample and (2a) respective reference amounts of the identified analytes and/or (2b) respective reference values of one or more analyte expressions, wherein each analyte expression includes a respective combination of at least two of the identified analytes, and wherein the profile data indicate the reference amounts and/or reference values; and
presenting the determined score for the aspect of the sample to a user via a user interface of a computer.
